(19) United States
(12) Patent Application Publication
(10) Pub. No.: US 2003/0148409 A1
(43) Pub. Date: Aug. 7, 2003

(54) Direct targeting binding proteins

(76) Inventors: Edmund Rossi, Nutley, NJ (US); Chien-Hsing Ken Chang, Downington, PA (US); David M. Goldenberg, Mendham, NJ (US)

Correspondence Address: FOLEY AND LARDNER, SUITE 500, 3000 K STREET NW, WASHINGTON, DC, 20007, US

(21) Appl. No.: 10/270,073

(22) Filed: Oct. 15, 2002

Related U.S. Application Data
(60) Provisional application No. 60/328,835, filed on Oct. 15, 2001.
(60) Provisional application No. 60/341,881, filed on Dec. 21, 2001.
(60) Provisional application No. 60/345,641, filed on Jan. 8, 2002.
(60) Provisional application No. 60/404,919, filed on Aug. 22, 2002.

(51) Int. Cl. A61K 51/00; G01N 33/574; C07K 16/30

(52) U.S. Cl. 435/7.23; 424/1.49; 530/388.8

(57) ABSTRACT

The present invention relates to multivalent, monospecific binding proteins. These binding proteins comprise two or more binding sites, where each binding site specifically binds to the same type of target cell, and preferably with the same antigen on such a target cell. The present invention further relates to compositions of monospecific diabodies, triabodies, and tetrabodies, and to recombinant vectors useful for the expression of these functional binding proteins in a microbial host. Also provided are methods of using invention compositions in the treatment and/or diagnosis of tumors.

### RELATED APPLICATIONS

This application claims the benefit of U.S. Provisional Applications 60/328,835, filed Oct. 15, 2001, 60/341,881 filed Dec. 21, 2001, 60/345,641 filed Jan. 8, 2002 and 60/404,919, filed Aug. 22, 2002, the contents of each of which are hereby incorporated herein in their entirety.

### FIELD OF THE INVENTION

The present invention relates generally to multivalent, monospecific binding proteins. In particular, the present invention relates to compositions of monospecific diabodies, triabodies, and tetrabodies and methods of use thereof, and to recombinant vectors useful for the expression of these functional binding proteins in a microbial host.

### BACKGROUND OF THE INVENTION

The following description is provided to assist the understanding of the reader. None of the information provided or references cited is admitted to be prior art to the present invention.

Man-made binding proteins, in particular, monoclonal antibodies and engineered antibodies or antibody fragments, have been tested widely and have been shown to be of value in the detection and treatment of various human disorders, including cancers, autoimmune diseases, infectious diseases, inflammatory diseases and cardiovascular diseases (Filpula and McGuire, Exp. Opin. Ther. Patents 9:231-245 (1999)). For example, antibodies labeled with radioactive isotopes have been used to visualize tumors using detectors available in the art, following their injection into a patient. The clinical utility of an antibody or an antibody-derived agent is primarily dependent on its ability to specifically bind to a target antigen. Selectivity is valuable for the effective delivery of a diagnostic or a therapeutic agent (such as drugs, toxins, cytokines, hormones, growth factors, conjugates, radionuclides, or metals) to a target location for the detection and/or treatment phases of a human disorder, particularly if the diagnostic or therapeutic agent is toxic to normal tissue in the body.

The potential limitations of antibody systems are known in the art (see, e.g., Goldenberg, Am. J. Med. 94:297-312 (1993)). Important parameters in detection and treatment techniques include, for example, the amount of the injected dose specifically localized at the site(s) where cells containing the target antigen are present and the uptake ratio, i.e. the ratio of the amount of specifically bound antibody to that of the free antibody present in surrounding normal tissues (as detected by radioactivity). When an antibody is injected into the blood stream, it passes through a number of physiological compartments as it is metabolized and excreted. Optimally, the antibody should be able to locate and bind to the target cell antigen while passing through the rest of the body. Factors that control antigen targeting include, for example, the location and size of the antigen, antigen density, antigen accessibility, the cellular composition of the target tissue, and the pharmacokinetics of the targeting antibodies. Other factors that specifically affect tumor targeting by antibodies include the expression levels of the target antigen, both in tumor and normal tissues, and bone marrow toxicity resulting from slow blood-clearance of radiolabeled antibodies.

The amount of targeting antibodies accreted by targeted tumor cells is influenced by vascularization of the tumor and barriers to antibody penetration of tumors, as well as intratumoral pressure. Non-specific uptake by non-target organs (such as the liver, kidneys or bone marrow) is another potential limitation of the technique, especially for radioimmunotherapy, where irradiation of the bone marrow often causes dose-limiting toxicity.

An approach referred to as,direct targeting, is designed to target tumor antigens using antibodies carrying a diagnostic or therapeutic radioisotope. The direct targeting approach requires a radiolabeled anti-tumor monospecific antibody that specifically recognizes a target antigen located on or within the tumor. The technique generally involves injecting the labeled monospecific antibody into the patient and allowing the antibody to localize, to the tumor to obtain diagnostic or therapeutic benefits, while unbound antibody clears the body. However, the radiolabeled antibody does not form a very stable complex with the target antigen, and therefore, does not remain at the tumor site for a long period of time.

Thus, there remains a need in the art for compositions of multivalent, monospecific antibodies and methods of producing such antibodies using recombinant DNA technology for use in a direct targeting system. Specifically, there remains a need for an antibody that exhibits enhanced antibody uptake and binding to target antigens, leaving less free antibody in the circulation, and optimal protection of normal tissues and cells from toxic agents complexed with the antibody.

### SUMMARY OF THE INVENTION

The present invention relates to multivalent, monospecific binding proteins. These binding proteins comprise two or more binding sites, where each binding site specifically binds to the same type of target cell, and preferably with the same antigen on such a target cell. The present invention further relates to compositions of monospecific diabodies, triabodies, and tetrabodies, and to recombinant vectors useful for the expression of these functional binding proteins in a microbial host. Also provided are methods of using invention compositions in the treatment and/or diagnosis of tumors. It is a specific object of the present invention to provide antibodies that exhibit enhanced antibody uptake and binding to target antigens, for use in the diagnosis and treatment of tumors.

According to one aspect of the present invention there are provided multivalent, monospecific-binding proteins which have two or more binding sites specific for the same target antigen. Each binding site is formed by the association of two or more single chain Fv (scFv) fragments, and each scFv comprises at least 2 variable domains derived from a humanized or human monoclonal antibody. In various alternative preferred embodiments, the multivalent, monospecific binding protein may be a monospecific diabody, a monospecific triabody, or a monospecific tetrabody. In preferred embodiments, the humanized or human monoclonal antibody is specific for a tumor-associated antigen, most preferably the carcinoembryonic antigen (CEA).

According to another aspect of the present invention, the multivalent, monospecific binding protein may also contain a diagnostic agent, a therapeutic agent, and/or combinations of two or more of such agents. In various embodiments, the diagnostic agent may be a conjugate, a radionuclide, a metal, a contrast agent, a tracking agent, a detection agents, or a combination thereof. In various embodiments, the therapeutic agent may be a radionuclide, a chemotherapeutic drug, a cytokine, a hormone, a growth factor, a toxin, an immunomodulator, or a combination thereof.

According to yet another aspect of the present invention there are provided expression vectors comprising nucleotide sequences that encode the various multivalent, monospecific binding proteins, as well as host cells that have been transformed with these expression vectors for the production of the binding proteins.

The present invention further provides methods of diagnosing the presence of a tumor and methods of treating a tumor using invention binding proteins. The binding proteins of the present invention also serve as an effective means of delivering one or more diagnostic agent, one or more therapeutic agent, or a combination of two or more thereof to a tumor in a subject; and may be conveniently provided in a kit for therapeutic and/or diagnostic use for practitioners.

### BRIEF DESCRIPTION OF THE DRAWINGS

FIG. 1 is a schematic representation of the hMN-14scFv polypeptide synthesized in E. coli from the hMN-14-scFv-L5 expression plasmid, and the formation of a hMN-14 diabody. The nucleic acid construct encoding the unprocessed polypeptide contains sequences encoding the pelB signal peptide, the hMN-14V_{H} and hMN-14V_{K} coding sequences coupled by a 5 amino acid linker, and a carboxyl terminal histidine affinity tag. The figure also shows a stick figure drawing of the mature polypeptide following proteolytic removal of the pelB signal peptide, and a stick figure drawing of a hMN-14 diabody, including CEA binding sites.

FIG. 2 collectively shows the results of size-exclusion high performance liquid chromatography (HPLC) analysis of hMN-14 diabody purification. FIG. 2A is the HPLC elution profile of IMAC-purified hMN-14 diabody. The HPLC elution peaks of hMN-14 diabody in FIGS. 2A and 2B are identified with an arrow. FIG. 2B is the HPLC elution profile of hMN-14 diabody purified by WI2 anti-idiotype affinity chromatography. The *9.75 indicated on the x-axis of FIG. B is the HPLC retention time (9.75 minutes) of control hMN-14-Fab′-S-NEM (MW ˜50 kDa).

FIG. 3 collectively shows the results of protein analysis of the hMN-14scFv polypeptide. FIG. 3A is a reducing SDS-PAGE gel stained with Coomassie blue illustrating the purity of the hMN-14 diabody samples following IMAC purification and WI2 anti-idiotype affinity purification. The positions of the molecular weight standards and the hMN-14scFv polypeptide are indicated with arrows. FIG. 3B is an isoelectric focusing (IEF) gel. The positions of pI standards and hMN-14scFv polypeptide are indicated with arrows. Lane 1 of FIG. 3B contains the hMN-14 Fab′-S-NEM used as a standard. Lane 2 of the same figure contains the WI2 purified hMN-14 diabody. Lane 3 contains the unbound flow-through fraction from the WI2 affinity column, which indicated that the hMN-14scFv diabody is effectively purified by this process.

FIG. 4 shows the level of ¹³¹I-hMN-14 diabody over the first 96 hours following injection of the diabody as monitored in tumor and blood samples. The amount of ¹³¹I-hMN-14 diabody, measured as the percentage of the injected dose per gram of tissue (%ID/g), is plotted against time. Solid squares mark the data points for tumor samples and open boxes mark those of blood samples.

FIG. 5 shows the biodistribution of ¹³¹I-hMN-14 diabody 48 hours following injection. Samples were taken from tumor and normal tissues, including liver, spleen, kidney, lung, blood, stomach, small intestine and large intestine. The amount of ¹³¹I-hMN-14 diabody is displayed as the percentage of the injected dose per gram of tissue (%ID/g).

FIG. 6 is a schematic representation of the hMN-14-0 polypeptide synthesized in E. coli from the hMN-14-0 expression plasmid, and the formation of a hMN-14 triabody. The nucleic acid construct encoding the unprocessed polypeptide contains sequences encoding the pelB signal peptide, the hMN-14 V_{H} and hMN-14V_{K} coding sequences, and a carboxyl terminal histidine affinity tag. The figure also shows a stick figure drawing of the mature polypeptide following proteolytic removal of the pelB signal peptide, and a stick figure drawing of a hMN-14 triabody, including CEA binding sites.

FIG. 7 shows the results of size-exclusion HPLC analysis of the hMN-14 triabody purification. The HPLC elution peak of hMN-14 triabody is at 9.01 minutes. Soluble proteins were purified by Ni-NTA IMAC followed by Q-Sepharose anion exchange chromatography. The flow-through fraction of the Q-Sepharose column was used for HPLC analysis. The retention times of hMN-14 diabody and hMN-14 F(ab′)₂ are indicated with arrows.

FIG. 8 collectively shows a comparison of tumor uptake and blood clearance of hMN-14 diabody (FIG. 8A), hMN-14 triabody (FIG. 8B) and hMN-14 tetrabody (FIG. 8C) over the first 96 hours following injection. The amount of ¹²⁵I-labeled proteins, measured as the percentage of the injected dose per gram of tissue (%ID/g), is plotted against time.

FIG. 9 is a schematic representation of the hMN-14-1G polypeptide synthesized in E. coli from the hMN-14-1G expression plasmid, and the formation of a hMN-14 tetrabody. The nucleic acid construct encoding the unprocessed polypeptide contains sequences encoding the pelB signal peptide, the hMN-14 V_{H} and V_{K} coding sequences coupled by a single glycine residue, and the carboxyl terminal histidine affinity tag. The figure also shows a stick figure drawing of the mature polypeptide following proteolytic removal of the pelB signal peptide, and a stick figure drawing of a hMN-14 tetrabody, including CEA binding sites.

FIG. 10 shows the results of size-exclusion HPLC analysis of the hMN-14-1G polypeptide purification. Soluble proteins were purified by Ni-NTA IMAC followed by Q-Sepharose anion exchange chromatography. The flow-through fraction of the Q-Sepharose column was used for HPLC analysis. The HPLC elution peaks of diabody, triabody and tetrabody are indicated with arrows.

FIG. 11 is the nucleic acid sequence (SEQ ID NO: 1) and the deduced amino acid sequence (SEQ ID NO: 2) of hMN-14-scFv-L5. Nucleic acid bases 1-66 encode the pelB signal peptide; 70-423 encode hMN-14 V_{H}; 424-438 encode the linker peptide (GGGGS); 439-759 encode hMN-14 V_{K}; and 766-783 encode the histidine affinity tag.

FIG. 12 is the deduced amino acid sequence of hMN-14 V_{H} (SEQ ID NO: 3) and of hMN-14 V_{K} (SEQ ID NO: 4).

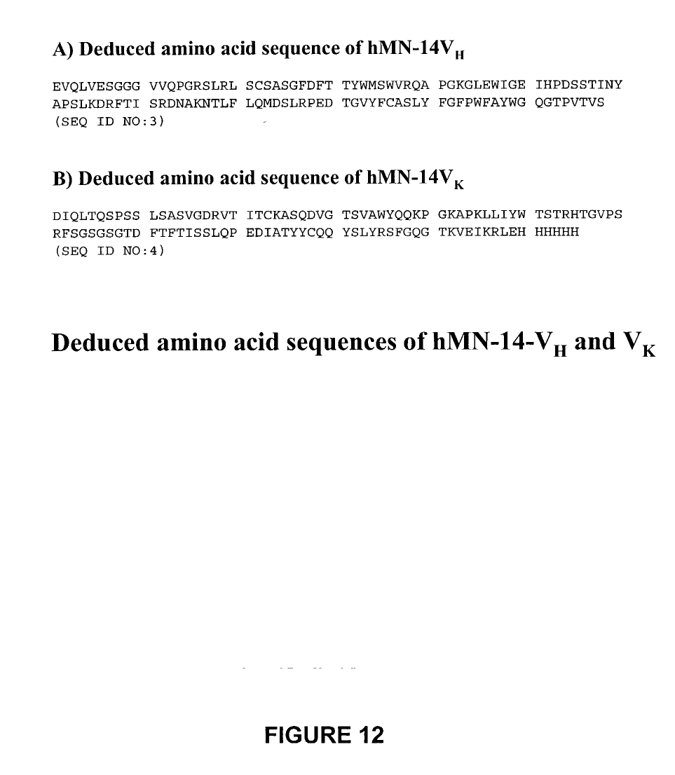

FIG. 13 is the nucleic acid sequence (SEQ ID NO: 5) and the deduced amino acid sequence (SEQ ID NO: 6) of hMN-14-0. Nucleic acid bases 1-66 encode the pelB signal peptide; 70-423 encode hMN-14V_{H}; 424-744 encode hMN-14V_{K}; and 751-768 encode the histidine affinity tag.

FIG. 14 is the nucleic acid sequence (SEQ ID NO: 7) and the deduced amino acid sequence (SEQ ID NO: 8) of hMN-14-1G. Nucleic acid bases 1-66 encode the pelB signal peptide; 70-423 encode hMN-14V_{H}; 424-427 encode the linker peptide (G); 427-747 encode hMN-14V_{K}; and 754-771 encode the histidine affinity tag.

### DETAILED DESCRIPTION OF THE PREFERRED EMBODIMENTS

Unless otherwise specified, “a” or “an” means “one or more”.

One embodiment of this invention relates to multivalent, monospecific binding proteins. These binding proteins comprise two or more binding sites where each binding site has affinity for the same single target antigen. Each binding site is formed by the association of two or more single chain Fv (scFv) fragments. Each scFv comprises at least two variable domains derived from a humanized or human monoclonal antibody. The present invention further relates to monospecific diabodies, triabodies, and tetrabodies, which may further comprise a diagnostic or therapeutic agent, or a combination of two or more thereof.

Accordingly, the present invention provides a multivalent, monospecific binding protein comprising two or more binding sites having affinity for the same single target antigen, wherein said binding sites are formed by the association of two or more single chain Fv (scFv) fragments, and wherein each scFv fragment comprises at least 2 variable domains derived from a humanized or human monoclonal antibody. In certain embodiments, said monoclonal antibody is specific for a tumor-associated antigen.

Structurally, whole antibodies are composed of one or more copies of an Y-shaped unit that contains four polypeptide chains. Two chains are identical copies of a polypeptide, referred to as the heavy chain, and two chains are identical copies of a polypeptide, referred to as the light chain. Each polypeptide is encoded by individual DNA or by connected DNA sequences. The two heavy chains are linked together by one or more disulfide bonds and each light chain is linked to one of the heavy chains by one disulfide bond. Each chain has an N-terminal variable domain, referred to as V_{H} and V_{L} for the heavy and-the light chains, respectively, and the non-covalent association of a pair of V_{H} and V_{L}, referred to as the Fv fragment, forms one antigen-binding site.

Discrete Fv fragments are prone to dissociation at low protein concentrations and under physiological conditions (Glockshuber et al., Biochemistry 29:1362-1367 (1990)), and therefore have limited use. To improve stability and enhance potential utility, recombinant single-chain Fv (scFv) fragments have been produced and studied extensively, in which the C-terminal of the V_{H} domain (or V_{L}) is joined to the N-terminal of the V_{L} domain (or V_{H}) via a peptide linker of variable length. (For a recent review, see Hudson and Kortt, J. Immunol. Meth. 231:177-189 (1999)).

ScFvs with linkers greater than 12 amino acid residues in length (for example, 15 or 18 residue linkers) allow interactions between the V_{H} and V_{L} regions of the same polypeptide chain and generally form a mixture of monomers, dimers (termed diabodies) and small amounts of higher mass multimers (Kortt et al., Eur. J. Biochem. 221:151-157 (1994)). ScFvs with linkers of 5 or less amino acid residues, however, prohibit intramolecular association of the V_{H} and V_{L} regions of the same polypeptide chain, forcing pairing with V_{H} and V_{L} domains on a different polypeptide chain. Linkers between 3 and 12 amino acid residues form predominantly dimers (Atwell et al., Prot. Eng. 12:597-604 (1999)). ScFvs with linkers between 0 and 2 amino acid residues form trimeric (termed triabodies), tetrameric (termed tetrabodies) or higher oligomeric structures; however, the exact patterns of oligomerization appear to depend on the composition as well as the orientation of the V-domains, in addition to the linker length. For example, scFvs of the anti-neuraminidase antibody NC10 form predominantly trimers (V_{H} to V_{L} orientation) or tetramers (V_{L} to V_{H} orientation) with 0 amino acid residue linkers (Dolezal et al., Prot. Eng. 13:565-574 (2000)). ScFvs constructed from NC10 with 1 and 2 amino acid residue linkers, in the V_{H} to V_{L} orientation, form predominantly diabodies (Atwell et al., supra); in contrast, the V_{L} to V_{H} orientation forms a mixture of tetramers, trimers, dimers, and higher mass multimers (Dolezal et al., supra). ScFvs constructed from the anti-CD19 antibody HD37, in the V_{H} to V_{L} orientation, with a 0 amino acid residue linker form exclusively trimers, while the same construct with a 1 amino acid residue linker forms exclusively tetramers (Le Gall et al., FEBS Lett. 453:164-168 (1999)).

The non-covalent association of two or more scFv molecules can form functional diabodies, triabodies and tetrabodies, which are multivalent but monospecific. Monospecific diabodies are homodimers of the same scFv, where each scFv comprises the V_{H} domain from the selected antibody connected by a short linker to the V_{L} domain of the same antibody. A diabody is a bivalent dimer formed by the non-covalent association of two scFvs, yielding two Fv binding sites. A triabody results from the formation of a trivalent trimer of three scFvs, yielding three binding sites, and a tetrabody is a tetravalent tetramer of four scFvs, resulting in four binding sites. Several monospecific diabodies have been made using an expression vector that contains a recombinant gene construct comprising V_{H1}-linker-V_{L1}. (See Holliger et al., Proc. Natl. Acad. Sci. USA 90:6444-6448 (1993); Atwell et al., Mol. Immunol. 33:1301-1312 (1996); Holliger et al., Nature Biotechnol. 15:632-636 (1997); Helfrich et al., Int. J. Cancer 76:232-239 (1998); Kipriyanov et al., Int. J. Cancer 77:763-772 (1998); Holliger et al., Cancer Res. 59:2909-2916 (1999)). Methods of constructing scFvs are disclosed in U.S. Pat. Nos. 4,946,778 and 5,132,405. Methods of producing multivalent, monospecific binding proteins based on scFv are disclosed in U.S. Pat. Nos. 5,837,242 and 5,844,094, and PCT Application WO98/44001.

A humanized antibody is a recombinant protein in which the CDRs from an antibody from one species; e.g., a rodent antibody, is transferred from the heavy and light variable chains of the rodent antibody into human heavy and light variable domains. The constant domains of the antibody molecule is derived from those of a human antibody.

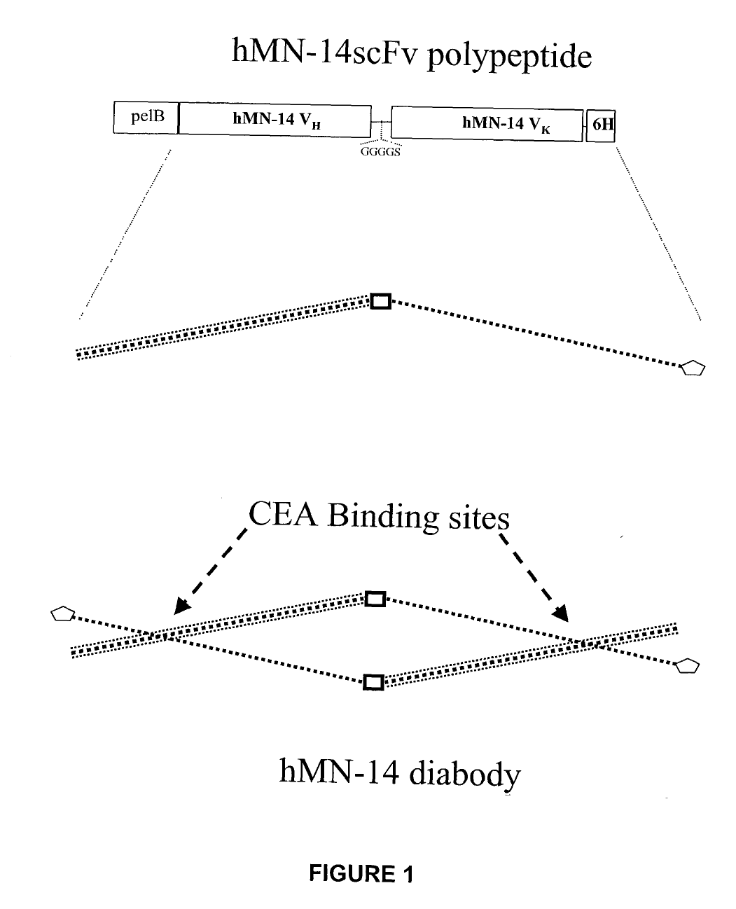

One embodiment of the present invention utilizes one monoclonal antibody, hMN-14, to produce antigen specific diabodies, triabodies, and tetrabodies. hMN-14 is a humanized monoclonal antibody (MAb) that binds specifically to CEA (Shevitz et al., J. Nucl. Med. S34:217 (1993); and U.S. Pat. No. 6,254,868). While the original MAbs were murine, humanized antibody reagents are now utilized to reduce the human anti-mouse antibody response. The variable regions of this antibody were engineered into an expression construct (hMN-14-scFv-L5) as described in Example 1. As depicted in FIG. 1, the nucleic acid construct (hMN-14-scFv-L5) for expressing an hMN-14 diabody encodes a polypeptide that possesses the following features:

(i) carboxyl terminal end of V_{H} linked to amino terminal end of V_{K} by the peptide linker Gly-Gly-Gly-Gly-Ser (G₄S) (the use of the G₄S peptide linker enables the secreted polypeptide to dimerize into a diabody, forming two binding sites for CEA);

(ii) pelb signal peptide sequence precedes the V_{H} gene to facilitate the synthesis of the polypeptide in the periplasmic space of E. coli; and

(iii) six histidine (6His) amino acid residues added to the carboxyl terminus to allow purification by IMAC.

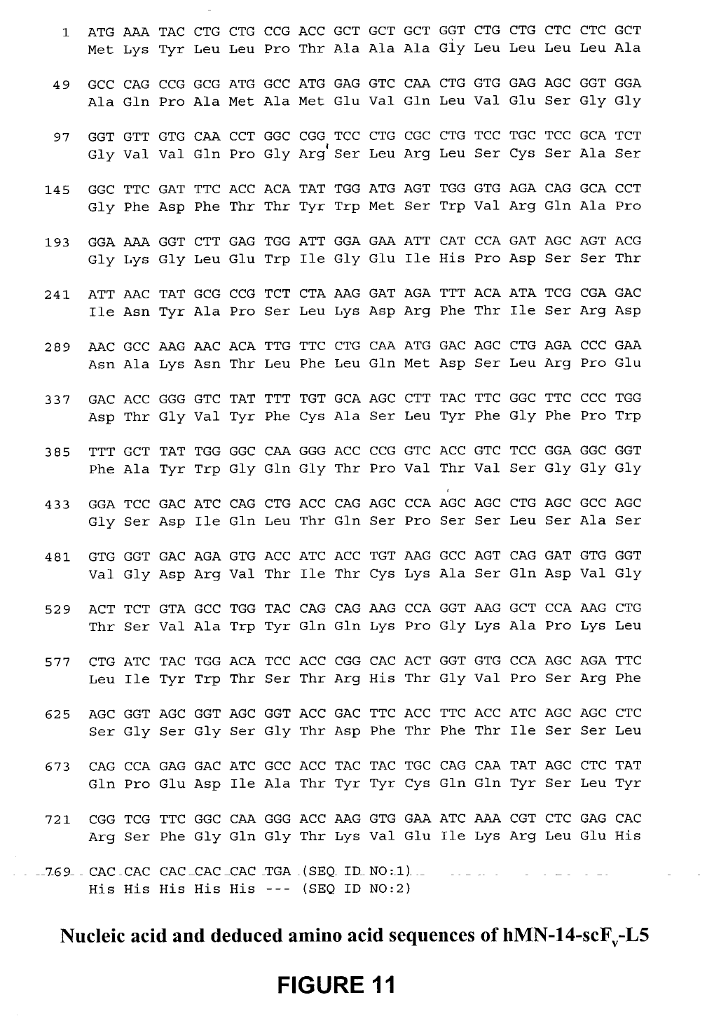

The coding sequence of the nucleic acid (SEQ ID NO: 1) and the corresponding deduced amino acid sequence (SEQ ID NO: 2) of hMN-14-scFv-L5 are presented in FIG. 11. FIG. 1 also shows a stick figure drawing of the mature polypeptide following proteolytic removal of the pelB signal peptide, and a stick figure drawing of a hMN-14 diabody, including CEA binding sites.

A human antibody is an antibody obtained from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994).

A fully human antibody also can be constructed by genetic or chromosomal transfection methods, as well as phage display technology, all of which are known in the art. See for example, McCafferty et al., Nature 348:552-553 (1990) for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see e.g. Johnson and Chiswell, Curr. Opin. Struct. Biol. 3:5564-571 (1993).

Human antibodies may also be generated by in vitro activated B cells. See U.S. Pat. Nos. 5,567,610 and 5,229,275, which are hereby incorporated by reference herein in their entirety.

Accordingly, the present invention provides a multivalent, monospecific binding protein comprising two binding sites having affinity for the same single target antigen (termed a monospecific diabody), wherein said binding sites are formed by the association of two single chain Fv (scFv) fragments, and wherein each scFv fragment comprises at least 2 variable domains derived from a humanized or human monoclonal antibody. In certain embodiments, said monoclonal antibody is specific for a tumor-associated antigen. Preferably, said tumor-associated antigen is carcinoembryonic antigen (CEA).

In further embodiments, the humanized monoclonal antibody of this monospecific diabody is hMN-14. In such an embodiment, each scFv preferably comprises the V_{H} and the V_{K} regions of hMN-14. Optionally, each scFv further comprises an amino acid linker connecting the V_{H} and the V_{K} regions of hMN-14. In a preferred embodiment, each scFv comprises the amino acid sequence of SEQ ID NO: 2.

Expression vectors were constructed through a series of sub-cloning procedures outlined in FIG. 1 and described in Example 2. The expression cassette for monospecific hMN-14 binding proteins is shown schematically in FIG. 1. The expression cassette may be contained in a plasmid, which is a small, double-stranded DNA forming an extra-chromosomal self-replicating genetic element in a host cell. A cloning vector is a DNA molecule that can replicate on its own in a microbial host cell. This invention describes vectors that expresses monospecific diabodies, triabodies, and tetrabodies. A host cell accepts a vector for reproduction and the vector replicates each time the host cell divides.

Accordingly, the present invention also provides an expression vector comprising a nucleotide sequence encoding a monospecific diabody as described.

A commonly used host cell is Escherichia coli (E. coli), however, other host cells are well known in the art, such as, for example, various bacteria, mammalian cells, yeast cells, and plant cells. In yeast, a number of vectors known to those of skill in the art can be used to introduce and express constructs in Saccharomyces cerevisiae (baker's yeast), Schizosaccharomyces pombe (fission yeast), Pichia pastoris, and Hansenula polymorpha (methylotropic yeasts). In addition, a variety of mammalian expression vectors are commercially available. Further, a number of viral-based expression systems, such as adenovirus and retroviruses, can be utilized. By using such an expression system, large quantities of recombinant antibody can be produced using methods of the present invention, enabling their use as a viable delivery system.

Accordingly, the present invention also provides a host cell comprising an expression vector encoding a monospecific diabody as described.

When the cassette as-shown in FIG. 1 is expressed in E. coli, some of the polypeptides fold and spontaneously form soluble monospecific diabodies. The monospecific diabody shown in FIG. 1 has two polypeptide chains that interact with each other to form two CEA binding sites having affinity for CEA antigens. Antigens are bound by specific antibodies to form antigen-antibody complexes, which are held together by the non-covalent interactions of antigen and antibody molecules.

In this embodiment, two polypeptides comprising the V_{H} region of the hMN-14 MAb connected to the V_{K} region of the hMN-14 MAb by a five amino acid residue linker are utilized. Each polypeptide forms one half of the hMN-14 diabody. The coding sequence of the nucleic acid (SEQ ID NO: 1) and the corresponding deduced amino acid sequence (SEQ ID NO: 2) of each polypeptide are presented in FIG. 11.

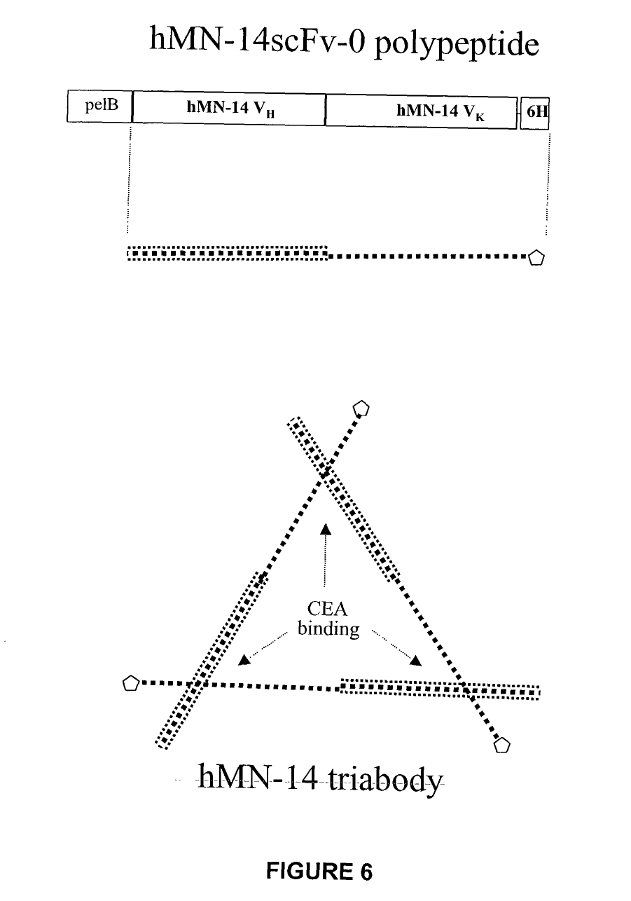

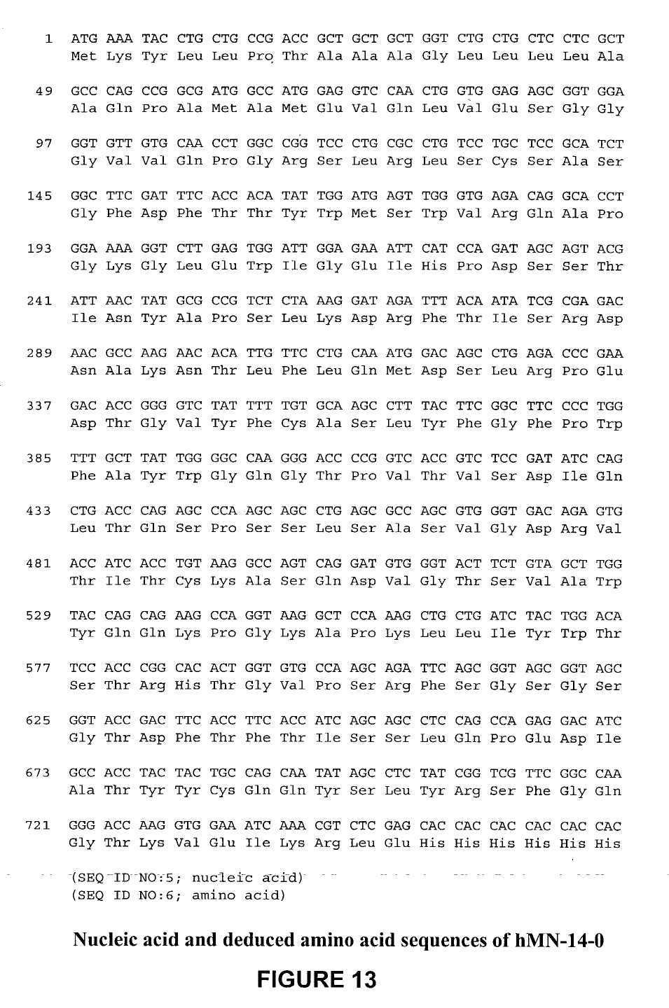

In the case of triabodies, when the cassette as shown in FIG. 6 is expressed in E. coli, some of the polypeptides spontaneously form soluble monospecific triabodies. The monospecific triabody shown in FIG. 6 has three polypeptide chains that interact with each other to form three CEA binding sites having high affinity for CEA antigens. Each of the three polypeptides comprise the V_{H} region of the hMN-14 MAb connected to the V_{K} region of the hMN-14 MAb, without a linker. Each polypeptide forms one third of the hMN-14 triabody. The coding sequence of the nucleic acid (SEQ ID NO: 5) and the corresponding deduced amino acid sequence (SEQ ID NO: 6) of each polypeptide is presented in FIG. 13.

Accordingly, the present invention provides a multivalent, monospecific binding protein comprising three binding sites having affinity for the same single target antigen (termed a monospecific triabody), wherein said binding sites are formed by the association of three single chain Fv (scFv) fragments, and wherein each scFv fragment comprises at least 2 variable domains derived from a humanized or human monoclonal antibody. In certain embodiments, said monoclonal antibody is specific for a tumor-associated antigen. Preferably, said tumor-associated antigen is carcinoembryonic antigen (CEA).

In further embodiments, the humanized monoclonal antibody of this monospecific triabody is hMN-14. In such an embodiment, each scFv preferably comprises the V_{H} and the V_{K} regions of hMN-14. In certain embodiments, each scFv comprises the amino acid sequence of SEQ ID NO: 6. The present invention also provides an expression vector comprising a nucleotide sequence encoding the monospecific triabody and a host cell comprising this expression vector.

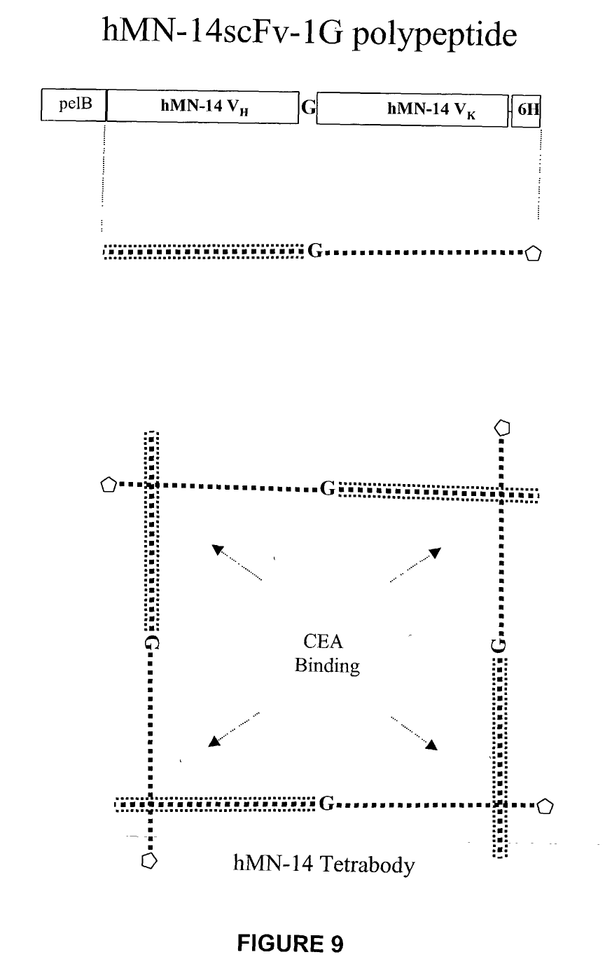

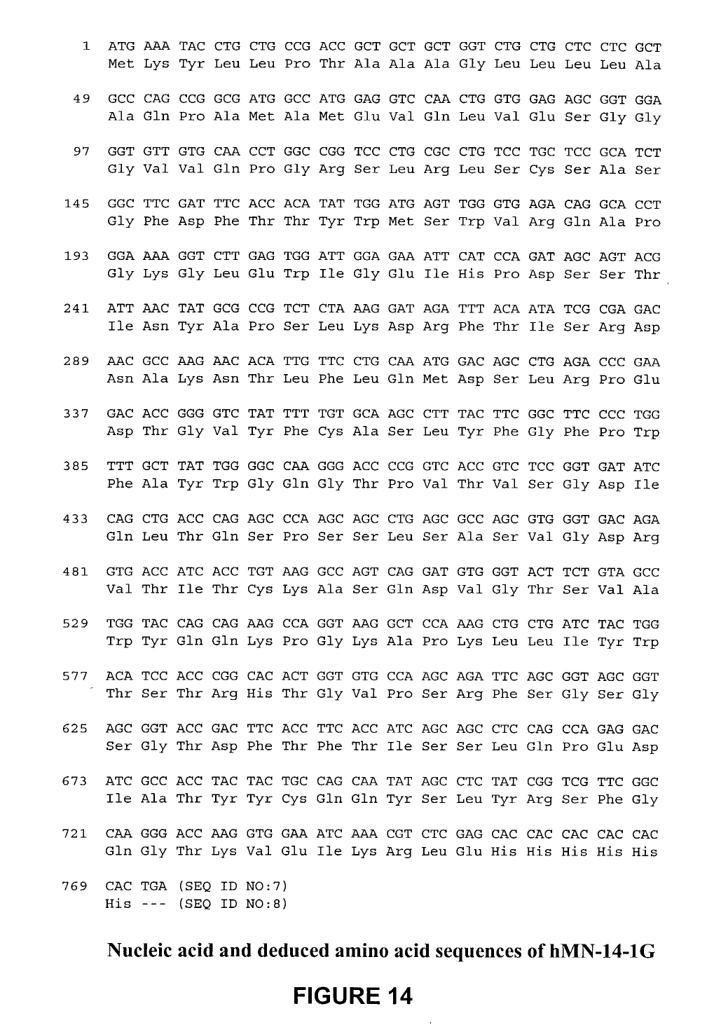

In the case of tetrabodies, when the cassette as shown in FIG. 9 is expressed in E. coli, some of the polypeptides spontaneously form soluble monospecific tetrabodies. The monospecific tetrabody shown in FIG. 9 has four polypeptide chains that interact with each other to form four CEA binding sites having high affinity for CEA antigens. Each of the four polypeptides comprise the V_{H} polypeptide of the hMN-14 MAb connected to the V_{K} polypeptide of the hMN-14 MAb by a single amino acid residue linker. Each polypeptide forms one fourth of the hMN-14 tetrabody. The coding sequence of the nucleic acid (SEQ ID NO: 7) and the corresponding deduced amino acid sequence (SEQ ID NO: 8) of each polypeptide is contained in FIG. 14.

Accordingly, the present invention provides a multivalent, monospecific binding protein comprising four binding sites having affinity for the same single target antigen (termed a monospecific tetrabody), wherein said binding sites are formed by the association of four single chain Fv (scFv) fragments, and wherein each scFv fragment comprises at least 2 variable domains derived from a humanized or human monoclonal antibody. In certain embodiments, said monoclonal antibody is specific for a tumor-associated antigen. Preferably, said tumor-associated antigen is carcinoembryonic antigen (CEA).

In further embodiments, the humanized monoclonal antibody is hMN-14. In such an embodiment, each scFv preferably comprises the V_{H} and the V_{K} regions of hMN-14. Optionally, each scFv further comprises an amino acid linker connecting the V_{H} and the V_{K} regions of hMN-14. In certain embodiments, each scFv comprises the amino acid sequence of SEQ ID NO: 8. The present invention also provides an expression vector comprising a nucleotide sequence encoding the monospecific tetrabody and a host cell comprising this expression vector.

In a preferred embodiment, the monospecific diabodies, triabodies, and tetrabodies of the present invention are used for direct targeting of diagnostic or therapeutic agents to CEA positive tumors. Other tumor-associated antigens may also be targeted, such as A3, A33, BrE3, CD1, CD1a, CD3, CD5, CD15, CD19, CD20, CD21, CD22, CD23, CD30, CD45, CD74, CD79a, CEA, CSAp, EGFR, EGP-1, EGP-2, Ep-CAM, Ba 733, HER2/neu, KC4, KS-1, KS1-4, Le-Y, MAGE, MUC1, MUC2, MUC3, MUC4, PAM-4, PSA, PSMA, RS5, S100, TAG-72, tenascin, Tn antigen, Thomson-Friedenreich antigens, tumor necrosis antigens, VEGF, 17-1A, an angiogenesis marker, a cytokine, an immunomodulator, an oncogene marker and an oncogene product. The monospecific molecules bind selectively to targeted antigens and as the number of binding sites on the molecule increases, the affinity for the target cell increases. A stronger affinity allows the compositions of the present invention to remain at the desired location containing the target antigen for a longer time. Moreover, free unbound antibody molecules are cleared from the body quickly, thereby minimizing exposure of normal tissues to potentially harmful agents.

Tumor-associated markers have been categorized by Herberman (see, e.g., Immunodiagnosis of Cancer, in THE CLINICAL BIOCHEMISTRY OF CANCER, Fleisher ed., American Association of Clinical Chemists, 1979) in a number of categories including oncofetal antigens, placental antigens, oncogenic or tumor virus associated antigens, tissue associated antigens, organ associated antigens, ectopic hormones and normal antigens or variants thereof. Occasionally, a sub-unit of a tumor-associated marker is advantageously used to raise antibodies having higher tumor-specificity, e.g., the beta-subunit of human chorionic gonadotropin (HCG) or the gamma region of carcinoembryonic antigen (CEA), which stimulate the production of antibodies having a greatly reduced cross-reactivity to non-tumor substances as disclosed in U.S. Pat. Nos. 4,361,644 and 4,444,744. Markers of tumor vasculature (e.g., VEGF), of tumor necrosis, of membrane receptors (e.g., folate receptor, EGFR), of transmembrane antigens (e.g., PSMA), and of oncogene products can also serve as suitable tumor-associated targets for antibodies or antibody fragments. Markers of normal cell constituents which are overexpressed on tumor cells, such as B-cell complex antigens, as well as cytokines expressed by certain tumor cells (e.g., IL-2 receptor in T-cell malignancies) are also suitable targets for the antibodies and antibody fragments of this invention.

The BrE3 antibody is described in Couto et al., Cancer Res. 55:5973s-5977s (1995). The EGP-1 antibody is described in U.S. Provisional Application No. 60/360,229, some of the EGP-2 antibodies are cited in Staib et al., Int. J. Cancer 92:79-87 (2001); and Schwartzberg et al., Crit. Rev. Oncol. Hematol. 40:17-24 (2001). The KS-1 antibody is cited in Koda et al., Anticancer Res. 21:621-627 (2001); the A33 antibody is cited in Ritter et al., Cancer Res. 61:6854-6859 (2001); Le(y) antibody B3 is described in Di Carlo et al., Oncol. Rep. 8:387-392 (2001); and the A3 antibody is described in Tordsson et al., Int. J. Cancer 87:559-568 (2000).

Also of use are antibodies against markers or products of oncogenes, or antibodies against angiogenesis factors, such as VEGF. VEGF antibodies are described in U.S. Pat. Nos. 6,342,221, 5,965,132 and 6,004,554, and are incorporated by reference in their entirety. Antibodies against certain immune response modulators, such as antibodies to CD40, are described in Todryk et al., J. Immunol. Meth. 248:139-147 (2001) and Turner et al., J. Immunol. 166:89-94 (2001). Other antibodies suitable for combination therapy include anti-necrosis antibodies as described in Epstein et al., see e.g., U.S. Pat. Nos. 5,019,368; 5,882,626; and 6,017,514.

Accordingly, the present invention provides multivalent, monospecific binding proteins as described, comprising at least 2 variable domains derived from a humanized or human monoclonal antibody specific for a tumor-associated antigen associated with a disease state selected from the group consisting of a carcinoma, a melanoma, a sarcoma, a neuroblastoma, a leukemia, a glioma, a lymphoma and a myeloma. Said tumor-associated antigen may be associated with a type of cancer selected from the group consisting of acute lymphoblastic leukemia, acute myelogenous leukemia, biliary, breast, cervical, chronic lymphocytic leukemia, chronic myelogenous leukemia, colorectal, endometrial, esophageal, gastric, head and neck, Hodgkin's lymphoma, lung, medullary thyroid, non-Hodgkin's lymphoma, ovarian, pancreatic, prostrate, and urinary bladder. Said tumor-associated antigen may be selected from the group consisting of A3, A33, BrE3, CD1, CD1a, CD3, CD5, CD15, CD19, CD20, CD21, CD22, CD23, CD30, CD45, CD74, CD79a, CEA, CSAp, EGFR, EGP-1, EGP-2, Ep-CAM, Ba 733, HER2/neu, KC4, KS-1, KS1-4, Le-Y, MAGE, MUC1, MUC2, MUC3, MUC4, PAM-4, PSA, PSMA, RS5, S100, TAG-72, tenascin, Tn antigen, Thomson-Friedenreich antigens, tumor necrosis antigens, VEGF, 17-1A, an angiogenesis marker, a cytokine, an immunomodulator, an oncogene marker and an oncogene product. In a preferred embodiment, said tumor-associated antigen is carcinoembryonic antigen (CEA). In a preferred embodiment, the humanized monoclonal antibody is hMN-14.

A further embodiment of the invention involves using the inventive antibody or antibody fragment for detection, diagnosing and/or treating diseased tissues (e.g., cancers), comprising administering an effective amount of a bivalent, trivalent, or tetravalent antibody or antibody fragment comprising at least two arms that specifically bind a targeted tissue.

Accordingly, the present invention provides multivalent, monospecific binding proteins as described, further comprising at least one agent selected from the group consisting of a diagnostic agent, a therapeutic agent, and combinations of two or more thereof. Said diagnostic agent may selected from the group consisting of a conjugate, a radionuclide, a metal, a contrast agent, a tracking agent, a detection agent, and combinations of two or more thereof.

In certain embodiments comprising a diagnostic radionuclide, said radionuclide is selected from the group consisting of ¹¹C, ¹³N, ¹⁵O, ³²P ⁵¹Mn, ⁵²Fe, ^{52m}Mn, ⁵⁵Co, ⁶²Cu, ⁶⁴Cu, ⁶⁷Cu, ⁶⁷Ga, ⁶⁸Ga, ⁷²As, ⁷⁵Br, ⁷⁶Br, ^{82m}Rb, ⁸³Sr, ⁸⁶Y, ⁸⁹Zr, ⁹⁰Y, ^{94m}Tc, ⁹⁴Tc, ^{99m}Tc, ¹¹⁰In, ¹¹¹In, ¹²⁰I, ¹²³I, ¹²⁴I, ¹²⁵I, ¹³¹I, ¹⁵⁴⁻¹⁵⁸Gd, ¹⁷⁷Lu, ¹⁸⁶Re, ¹⁸⁸Re, a gamma-emitter, a beta-emitter, a positron-emitter, and combinations of two or more thereof. In certain other embodiments, said radionuclide is selected from the group consisting of ⁵Cr ⁵⁷Co, ⁵⁸Co, ⁵⁹Fe, ⁶⁷Cu, ⁶⁷Ga, ⁷⁵Se ⁹⁷Ru, ^{99m}Tc, ¹¹¹In, ^{114m}In, ¹²³I, ¹²⁵I, ¹³¹I, ¹⁶⁹Yb, ¹⁹⁷Hg, ²⁰¹Tl, and combinations of two or more thereof.

In certain embodiments comprising a metal, said metal is selected from the group consisting of gadolinium, iron, chromium, copper, cobalt, nickel, dysprosium, rhenium, europium, terbium, holmium, neodymium, and combinations of two or more thereof.

In certain embodiments comprising a contrast agent, said contrast agent may be a MRI contrast agent, a CT contrast agent, or an ultrasound contrast agent. A contrast agent may be selected from the group consisting of agadolinium ions, lanthanum ions, manganese ions, iron, chromium, copper, cobalt, nickel, dysporsium, rhenium, europium, terbium, holmium, neodymium, another comparable contrast agent, and combinations of two or more thereof.

In certain embodiments comprising a tracking agent, said tracking agent is selected from the group consisting of iodine compounds, barium compounds, gallium compounds, thallium compounds, barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, and combinations of two or more thereof.

In certain embodiments comprising a detection agent, said detection agent is selected from the group consisting of an enzyme, a fluorescent compound, a chemiluminescent compound, a bioluminescent compound, a radioisotope, and combinations of two or more thereof.

In certain embodiments comprising a therapeutic agent, said therapeutic agent is selected from the group consisting of a radionuclide, a chemotherapeutic drug, a cytokine, a hormone, a growth factor, a toxin, an immunomodulator, and combinations of two or more thereof.

In certain embodiments comprising a therapeutic radionuclide is selected from the group consisting of ³²p, ³³p, ⁴⁷Sc, ⁵⁹Fe, ⁶²Cu, ⁶⁴Cu, ⁶⁷Cu, ⁶⁷Ga, ⁷⁵Se, ⁷⁷As, ⁸⁹Sr, ⁹⁰Y, ⁹⁹Mo, ¹⁰⁵Rh, ¹⁰⁹Pd, ¹¹¹Ag, ¹¹¹In, ¹²⁵I, ¹³¹I, ¹⁴²Pr, ¹⁴³Pr, ¹⁴⁹Pm, ¹⁵³Sm, ¹⁶¹Tb, ¹⁶⁶Dy, ¹⁶⁶Ho, ¹⁶⁹Er, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, and combinations of two or more thereof. In certain other embodiments, said radionuclide is selected from the group consisting of ⁵⁸Co, ⁶⁷Ga, ⁸⁰mBr, ^{99m}Tc, ^{103m}Rh, ¹⁰⁹Pt, ¹¹¹In, ¹¹⁹Sb, ¹²⁵I, ¹⁶¹Ho, ^{189m}Os and ¹⁹²Ir, ¹⁵²Dy, ²¹At, ²¹¹Bi, ²¹²Bi, ²¹³Bi, ²¹⁵Po, ²¹⁷At, ²¹⁹Rn, ²²¹Fr, ²²³Ra, ²²⁵Ac, ²⁵⁵Fm, and combinations of two or more thereof.

In certain embodiments comprising a chemotherapeutic drug, said chemotherapeutic drug is selected from the group consisting of vinca alkaloids, anthracyclines, epidophyllotoxins, taxanes, antimetabolites, alkylating agents, antibiotics, Cox-2 inhibitors, antimitotics, antiangiogenic agents, apoptotoic agents, doxorubicin, methotrexate, taxol, CPT-11, camptothecans, nitrogen mustards, alkyl sulfonates, nitrosoureas, triazenes, folic acid analogs, pyrimidine analogs, purine analogs, platinum coordination complexes, hormones, and combinations of two or more thereof.

In certain embodiments comprising a toxin, said toxin is selected from the group consisting of ricin, abrin, ribonuclease, DNase I, Staphylococcal enterotoxin A, pokeweed antiviral protein, gelonin, diphtherin toxin, Pseudomonas exotoxin, Pseudomonas endotoxin, and combinations of two or more thereof.

In certain embodiments comprising an immunomodulator, said immunomodulator is selected from the group consisting of cytokines, stem cell growth factors, lymphotoxins, hematopoietic factors, colony stimulating factors, interferons, stem cell growth factors, erythropoietin, thrombopoietin, and combinations of two or more thereof.

A wide variety of diagnostic and therapeutic reagents can be advantageously conjugated to the antibodies of the invention. The therapeutic agents recited here are those agents that also are useful for administration separately with the multivalent binding proteins of the present invention as described herein. Therapeutic agents include, for example, chemotherapeutic drugs such as vinca alkaloids, anthracyclines, epidophyllotoxins, taxanes, antimetabolites, alkylating agents, antibiotics, Cox-2 inhibitors, antimitotics, antiangiogenic and apoptotoic agents, particularly doxorubicin, methotrexate, taxol, CPT-11, camptothecans, and others from these and other classes of anticancer agents, and the like. Other useful cancer chemotherapeutic drugs for the preparation of immunoconjugates and antibody fusion proteins include nitrogen mustards, alkyl sulfonates, nitrosoureas, triazenes, folic acid analogs, COX-2 inhibitors, pyrimidine analogs, purine analogs, platinum coordination complexes, hormones, and the like. Useful therapeutic combinations may comprise other agents used to treat CEA-producing cancers, anti-HER2 antibodies (e.g., Herceptin), and anti-EGF antibodies. Antibodies for combined use with the multivalent binding proteins of the present invention may be monoclonal, polyclonal, or humanized antibodies. Further suitable chemotherapeutic agents are described in Remington's Pharmaceutical ScienceS, 19th Ed. (Mack Publishing Co. 1995), and in Goodman and Gilman's the Pharmacological Basis of Therapeutics, 7th Ed. (MacMillan Publishing Co. 1985), as well as revised editions of these publications. Other suitable therapeutic agents, include experimental drugs and drugs involved in clinical trials, as are known to those of skill in the art.

A toxin, such as Pseudomonas exotoxin, may also be complexed to or form the therapeutic agent portion of an immunoconjugate of the antibodies of the present invention. Other toxins suitably employed in the preparation of such conjugates or other fusion proteins, include ricin, abrin, ribonuclcease (RNase), DNase I, Staphylococcal enterotoxin-A, pokeweed antiviral protein, gelonin, diphtherin toxin, Pseudomonas exotoxin, and Pseudomonas endotoxin (see, for example, Pastan et al., Cell 47:641-648 (1986), and Goldenberg, Calif. Cancer J. Clin. 44:43964 (1994)). Additional toxins suitable for use in the present invention are known to those of skill in the art and are disclosed in U.S. Pat. No. 6,077,499, which is incorporated in its entirety by reference.

The diagnostic and therapeutic agents can include drugs, toxins, cytokines, conjugates with cytokines, hormones, growth factors, conjugates, radionuclides, contrast agents, metals, cytotoxic drugs, and immune modulators. For example, gadolinium metal is used for magnetic resonance imaging and fluorochromes can be conjugated for photodynamic therapy. Moreover, contrast agents can be MRI contrast agents, such as gadolinium ions, lanthanum ions, manganese ions, iron, chromium, copper, cobalt, nickel, dysprosium, rhenium, europium, terbium, holmium, neodymium or other comparable label, CT contrast agents, and ultrasound contrast agents.

In the methods of the invention, the targetable construct may comprise one or more radioactive isotopes useful for detecting diseased tissue. Particularly useful diagnostic radionuclides include, but are not limited to, ¹¹C, ¹³N, ¹⁵O, ¹⁸F, ³²P, ⁵¹Mn, ⁵²Fe, ^{52m}Mn, ⁵⁵Co, ⁶²Cu, ⁶⁴Cu, ⁶⁷Cu, ⁶⁷Ga, ⁶⁸Ga, ⁷²As, ⁷⁵Br, ⁷⁶Br, ^{82m}Rb, ⁸³Sr, ⁸⁶Y, ⁸⁹Zr, ⁹⁰Y, ^{94m}Tc, ⁹⁴Tc, ^{99m}Tc, ¹¹⁰In, ¹¹¹In, ¹²⁰I, ¹²³I, ¹²⁴I, ¹²⁵I, ¹³¹I, ¹⁵⁴⁻¹⁵⁸Gd, ¹⁷⁷Lu, ¹⁸⁶Re, ¹⁸⁸Re, or other gamma-, beta-, or positron-emitters, preferably with a decay energy in the range of 20 to 4,000 keV, more preferably in the range of 25 to 4,000 keV, and even more preferably in the range of 20 to 1,000 keV, and still more preferably in the range of 70 to 700 keV. Total decay energies of useful positron-emitting radionuclides are preferably <2,000 keV, more preferably under 1,000 keV, and most preferably <700 keV.

Radionuclides useful as diagnostic agents utilizing gamma-ray detection include, but are not limited to: ⁵¹Cr, ⁵⁷Co, ⁵⁸Co, ⁵⁹Fe, ⁶⁷Cu, ⁶⁷Ga, ⁷⁵Se, ⁹⁷Ru, ^{99m}Tc, ¹¹¹In, ^{114m}In, ¹²³I, ¹²⁵I, ¹³¹I, ¹⁶⁹Yb, ¹⁹⁷Hg, and ²⁰¹Tl. Decay energies of useful gamma-ray emitting radionuclides are preferably 20-2000 keV, more preferably 60-600 keV, and most preferably 100-300 keV.

In the methods of the invention, the targetable construct may comprise one or more radioactive isotopes useful for treating diseased tissue. Particularly useful therapeutic radionuclides include, but are not limited to, ³²P, ³³P, 47Sc, ⁵⁹Fe, ⁶²Cu, ⁶⁴Cu, ⁶⁷Cu, ⁶⁷Ga, ⁷⁵Se, ⁷⁷As, ⁸⁹Sr, ⁹⁰Y, ⁹⁹Mo, ¹⁰⁵Rh, ¹⁰⁹Pd, ¹¹¹Ag, ¹¹¹In, ¹²⁵I, ¹³¹I, ¹⁴²Pr, ¹⁴³Pr ¹⁴⁹Pm, 153Sm, ¹⁶¹Tb, ¹⁶⁶Dy, ¹⁶⁶Ho, 169Er, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. The therapeutic radionuclide preferably has a decay energy in the range of 20 to 6,000 keV, preferably in the ranges 60 to 200 keV for an Auger emitter, 100-2,500 keV for a beta emitter, and 4,000-6,000 keV for an alpha emitter.

Also preferred are radionuclides that substantially decay with Auger-emitting particles. Such radionuclides include, but are not limited, ⁵⁸Co, ⁶⁷Ga, ^{80m}Br, ^{99m}Tc, ^{103m}Rh, ¹⁰⁹Pt, ¹¹¹In, ¹¹⁹Sb, ¹²⁵I, ¹⁶¹Ho, ^{189m}Os and ¹⁹²Ir. Also preferred are radionuclides that substantially decay with generation of alpha-particles. Such radionuclides include, but are not limited to, ¹⁵²Dy, ²¹¹At, ²¹¹Bi, ²¹²Bi, ²¹³Bi, ²¹⁵Po, ²¹⁷At, ²¹⁹Rn, ²²¹Fr, ²²³Ra, ²²⁵Ac and ²⁵⁵Fm. Decay energies of useful alpha-particle-emitting radionuclides are preferably 2,000-9,000 keV, more preferably 3,000-8,000 keV, and most preferably 4,000-7,000 keV.

The present invention antibodies and fragments thereof may include additional tracking agents. Radiopaque and contrast materials are used for enhancing X-rays and computed tomography, and include iodine compounds, barium compounds, gallium compounds, thallium compounds, etc. Specific compounds include barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride.

The present invention antibodies and fragments thereof also can be labeled with a fluorescent compound. The presence of a fluorescently-labeled MAb is determined by exposing the target antigen binding protein to light of the proper wavelength and detecting the resultant fluorescence. Fluorescent labeling compounds include fluorescein isothiocyanate, rhodamine, phycoerytherin, phycocyanin, allophycocyanin, o-phthaldehyde and fluorescamine. Fluorescently-labeled antigen binding proteins are particularly useful for flow cytometry analysis.

Alternatively, antibodies and fragments thereof can be detectably labeled by coupling the binding protein to a chemiluminescent compound. The presence of the chemiluminescent-tagged MAb is determined by detecting the presence of luminescence that arises during the course of a chemical reaction. Examples of chemiluminescent labeling compounds include luminol, isoluminol, an aromatic acridinium ester, an imidazole, an acridinium salt and an oxalate ester.

Similarly, a bioluminescent compound can be used to label antibodies and fragments thereof. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and aequorin.

Alternatively, antibodies and fragments thereof can be detectably labeled by linking the antibody to an enzyme. When the antibody-enzyme conjugate is incubated in the presence of the appropriate substrate, the enzyme moiety reacts with the substrate to produce a chemical moiety that can be detected, for example, by spectrophotometric, fluorometric or visual means. Examples of enzymes that can be used to detectably label antibody include malate dehydrogenase, staphylococcal nuclease, delta-V-steroid isomerase, yeast alcohol dehydrogenase, α-glycerophosphate dehydrogenase, triose phosphate isomerase, horseradish peroxidase, alkaline phosphatase, asparaginase, glucose oxidase, β-galactosidase, ribonuclease, urease, catalase, glucose-6-phosphate dehydrogenase, glucoamylase and acetylcholinesterase.

An immunomodulator, such as a cytokine, may also be conjugated to, or form the therapeutic agent portion of the antibody immunoconjugate, or be administered unconjugated to the chimeric, humanized, or human antibodies or fragments thereof of the present invention. As used herein, the term “immunomodulator” includes cytokines, stem cell growth factors, lymphotoxins, such as tumor necrosis factor (TNF), and hematopoietic factors, such as interleukins (e.g., interleukin-1 (IL-1), IL-2, IL-3, IL-6, IL-10, IL-12 and IL-18), colony stimulating factors (e.g., granulocyte-colony stimulating factor (G-CSF) and granulocyte macrophage-colony stimulating factor (GM-CSF)), interferons (e.g., interferons-α, -β and -γ), the stem cell growth factor designated “S1 factor,” erythropoietin and thrombopoietin. Examples of suitable inmmunomodulator moieties include IL-2, IL-6, IL-10, IL-12, IL-18, interferon-γ, TNF-α, and the like. Alternatively, subjects can receive naked antibodies and a separately administered cytokine, which can be administered before, concurrently or after administration of the naked antibodies. The antibody may also be conjugated to the immunomodulator. The immunomodulator may also be conjugated to a hybrid antibody consisting of one or more antibodies binding to different antigens.

A therapeutic or diagnostic agent can be attached at the hinge region of a reduced antibody component via disulfide bond formation. As an alternative, such peptides can be attached to the antibody component using a heterobifunctional cross-linker, such as N-succinyl 3-(2-pyridyldithio)proprionate (SPDP) (Yu et al., Int. J. Cancer 56: 244-248 (1994)). General techniques for such conjugation are well-known in the art. See, for example, Wong, Chemidtry of Protein Conjugstions and Cross-Linking RC Press 1991); Upeslacis et al., “Modification of Antibodies by Chemical Methods,” in Monoclonal Antibodies: Principles and Applications, Birch et al. (eds.), pages 187-230 (Wiley-Liss, Inc. 1995); Price, “Production and Characterization of Synthetic Peptide-Derived Antibodies,” in Monoclonal Antibodies: Production, Engineering and Clinical Application, Ritter et al. (eds.), pages 60-84 (Cambridge University Press 1995). Alternatively, the therapeutic or diagnostic agent can be conjugated via a carbohydrate moiety in the Fc region of the antibody. The carbohydrate group can be used to increase the loading of the same peptide that is bound to a thiol group, or the carbohydrate moiety can be used to bind a different peptide.

These agents are designed to diagnose and/or treat disorders in mammals. Mammals can include humans, domestic animals, and pets, such as cats and dogs. The mammalian disorders can include cancers, such as carcinomas, melanomas, sarcomas, neuroblastomas, leukemias, gliomas and myelomas. Exemplary types of cancers include, but are not limited to, biliary, breast, cervical, colorectal, endometrial, esophageal, gastric, head and neck, lung, medullary thyroid, ovarian, pancreatic, prostrate and urinary bladder.

Accordingly, the present invention provides a method of diagnosing the presence of a tumor, said method comprising administering to a subject suspected of having a tumor a detectable amount of a multivalent, monospecific binding protein as described, comprising a diagnostic agent as described, and monitoring the subject to detect any binding of the binding protein to a tumor.

The present invention further provides a method of treating a tumor, said method comprising administering to a subject in need thereof an effective amount of a multivalent, monospecific binding protein as described, comprising a diagnostic agent as described.

The present invention further provides a method of diagnosing the presence of a tumor, said method comprising administering to a subject suspected of having a tumor a detectable amount of a multivalent, monospecific binding protein as described, in combination with a detectable moiety that is capable of binding to said binding protein, and monitoring the subject to detect any binding of the binding protein to a tumor.

The present invention further provides a method of treating a tumor, said method comprising administering to a subject in need thereof an effective amount of a multivalent, monospecific binding protein as described, in combination with a therapeutic agent. In preferred embodiments, said therapeutic agent is selected from the group consisting of a chemotherapeutic drug, a toxin, external radiation, a brachytherapy radiation agent, a radiolabeled protein, an anticancer drug and an anticancer antibody.

The present invention further provides a method of delivering one or more diagnostic agent, one or more therapeutic agent, or a combination of two or more thereof to a tumor, said method comprising administering to a subject in need thereof a multivalent, monospecific binding protein as described, further comprising at least one agent selected from the group consisting of a diagnostic agent, a therapeutic agent, and combinations of two or more thereof.

Delivering a diagnostic or a therapeutic agent to a target for diagnosis or treatment in accordance with the invention includes providing the binding protein with a diagnostic or therapeutic agent and administering to a subject in need thereof with the binding protein. Diagnosis further requires the step of detecting the bound proteins with known techniques.

Administration of the binding protein with diagnostic or therapeutic agents of the present invention to a mammal may be intravenous, intraarterial, intraperitoneal, intramuscular, subcutaneous, intrapleural, intrathecal, by perfusion through a regional catheter, or by direct intralesional injection. When administering the binding protein by injection, the administration may be by continuous infusion or by single or multiple boluses.

The binding protein with the diagnostic or therapeutic agent may be provided as a kit for human or mammalian Aherapeutic and diagnostic use in a pharmaceutically acceptable injection vehicle, preferably phosphate-buffered saline (PBS) at physiological pH and concentration. The preparation preferably will be sterile, especially if it is intended for use in humans. Optional components of such kits include stabilizers, buffers, labeling reagents, radioisotopes, paramagnetic compounds, second antibody for enhanced clearance, and conventional syringes, columns, vials, and the like.

Accordingly, the present invention also provides a kit for therapeutic and/or diagnostic use, said kit comprising at least one multivalent, monospecific binding protein as described, further comprising at least one agent selected from the group consisting of a diagnostic agent, a therapeutic agent, and combinations of two or more thereof, and additional reagents, equipment, and instructions for use.

### EXAMPLES

The examples below are illustrative of embodiments of the current invention and should not be used, in any way, to limit the scope of the claims.

### Example 1

### Construction of Plasmids for Expression of hMN-14 Diabody in E. coli

Standard recombinant DNA methods were used to obtain hMN-14-scFv-L5 as follows. The hMN-14 V_{H} and V_{K} sequences were amplified from a vector constructed for expressing hMN-14 Fab′ (Leung et al., Cancer Res. 55:5968s-5972s (1995)) using the polymerase chain reaction (PCR) with Pfu polymerase. The hMN-14V_{H} sequence was amplified using the oligonucleotide primers specified below:

| | |
|---|---|
| hMN-14V_{H}-Left | (SEQ ID NO:9) |

5′ - CGTACCATGGAGGTCCAACTGGTGGAGA - 3′

| | |
|---|---|
| hMN-14V_{H}-Right (G₄S) | (SEQ ID NO:10) |

5′-CATAGGATCCACCGCCTCCGGAGACGGTGACCGGGGT - 3′

The left PCR primer contains a 5′ NcoI restriction site. The right PCR primer contains a sequence for a 5 amino acid residue linker (G₄S) and a BaniHI restriction site. The PCR product was digested with NcoI and BamHI and ligated, in frame with the pelB signal peptide sequence, into NcoI/BamHI digested pET-26b vector to generate hMN-14V_{H}L5-pET26. The hMN-14V_{K} sequence was amplified using the oligonucleotide primers specified below:

| | |
|---|---|
| hMN-14V_{K}-Left | (SEQ ID NO:11) |

5′ - CTGAGGATCCGACATCCAGCTGACCCAGAG - 3′

| | |
|---|---|
| hMN-14V_{K}-Right | (SEQ ID NO:12) |

5′ - GCTACTCGAGACGTTTGATTTCCACCTTGG - 3′

The left and right PCR primers contain BamHI and XhoI restriction sites, respectively. The PCR product was digested with XhoI and BamHI and ligated, in frame with the hMN-14V_{H}, G₄S linker and 6His sequences, into the XhoI/BamHI digested hMN-14VHL5-pET26 construct to generate the expression construct hMN-14-scFv-L5. The DNA sequence of this construct was verified by automated DNA sequencing, and is listed in FIG. 11. The nucleic acid construct, hMN-14-scFv-L5, is illustrated in FIG. 1.

### Example 2

### Expression of hMN-14 Diabody in E. coli

The hMN-14-scFv-L5 construct was used to transform BL21(P-LysS) E. coli. Culture conditions, induction, and purification were carried as described below. Competent E. coli BL21(P-Lys-S) cells were transformed with hMN-14-scFv-L5 by standard methods. Cultures were shaken in 2×YT media supplemented with 100 μg/ml kanamycin sulphate and 34 μg/ml chloramphenicol and grown at 37° C. to OD₆₀₀ of 1.6-1.8. An equal volume of room temperature 2×YT media supplemented with antibiotics and 0.8 M sucrose was added to the cultures, which were then transferred to 20° C. After 30 minutes at 20° C., expression was induced by the addition of 40 μM IPTG and continued at 20° C. for 15-18 hours.

The expression of hMN-14 diabody was examined in (1) cell culture conditioned media; (2) soluble proteins extracted under non-denaturing conditions from the cell pellet following centrifugation; and (3) insoluble material remained in the pellet following several cycles of extraction and centrifugation.

Soluble proteins were extracted from bacterial cell pellets as follows. Pellets were frozen and thawed, then re-suspended in lysis buffer (2% Triton X-100; 300 mM NaCl; 10 mM imidazole; 5 mM MgSO₄; 25 units/ml benzonase; 50 mM NaH₂PO₄ (pH 8.0)) using a volume equal to 1% of the culture volume. The suspension was homogenized by sonication, clarified by centrifugation, and loaded onto Ni-NTA IMAC columns. After being washed with buffer containing 20 mM imidazole, the columns were eluted with 100 mM imidazole buffer (100 mM imidazole; 50 mM NaCl; 25 mM Tris (pH 7.5)) and the eluate was further purified by affinity chromatography via binding to an anti-id antibody immobilized on Affi-gel.

The insoluble pelleted material was solubilized in denaturing Ni-NTA binding buffer (8 M urea; 10 mM imidazole; 0.1 M NaH₂PO₄; 10 mM Tris (pH 8.0)) and mixed with 1 ml of Ni-NTA agarose (Qiagen, Inc.). The mixture was rocked at room temperature for 1 hour, then the resin was washed once with 50 ml of the same buffer and loaded onto a column. The column was washed with 20 ml of the same buffer followed by 20 ml of wash buffer (8 M urea; 20 mM imidazole; 0.1 M NaH₂PO₄; 10 mM Tris (pH 8.0)). Bound proteins were eluted with 5 ml of denaturing elution buffer (8 M urea; 250 mM imidazole; 0.1 M NaH₂PO₄; 10 mM Tris (pH 8.0)).

Soluble proteins that bound to and were eluted from Ni-NTA resin were loaded on a WI2 anti-idiotype affinity column. The column was washed with PBS and the bound polypeptides were eluted with 0.1 M glycine; 0.1 M NaCl (pH 2.5) and neutralized immediately.

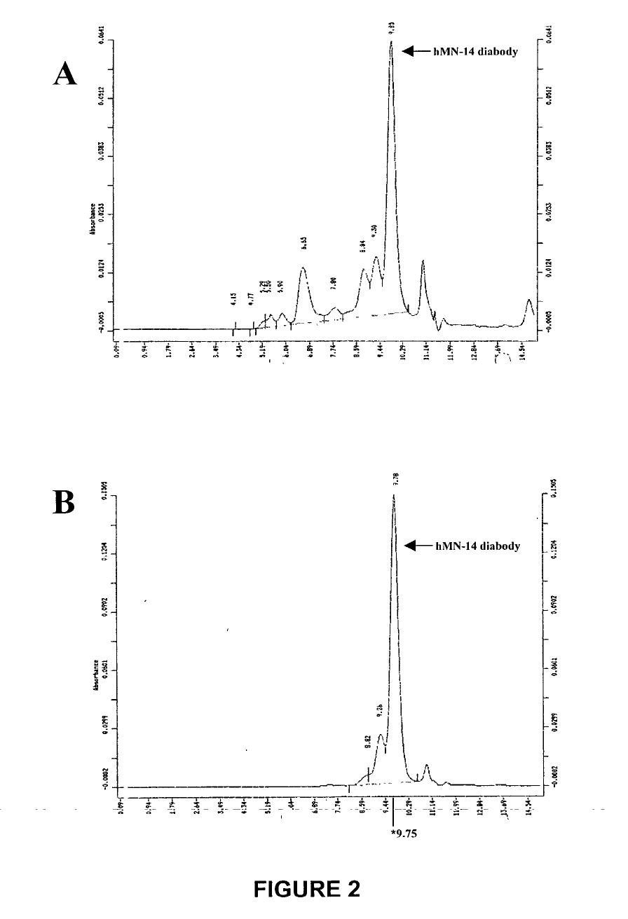

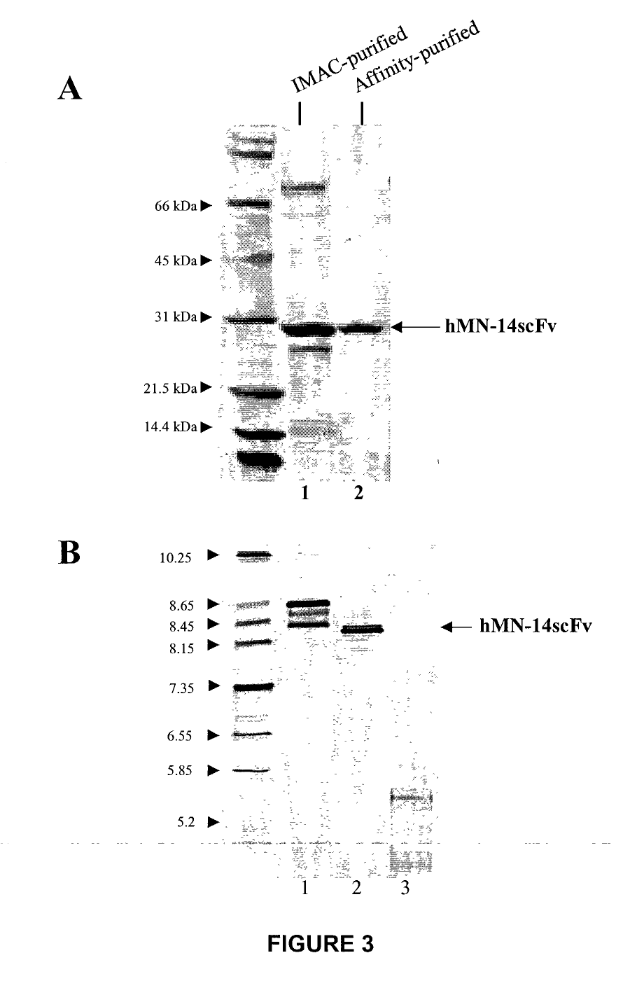

Although most of the hMN-14scFv expressed was present as insoluble protein, approximately 1.5 mg/L culture of soluble hMN-14scFv was purified from the soluble fraction. As shown by size-exclusion high performance liquid chromatography (HPLC), a predominant peak was observed (see FIGS. 2A and 2B) at 9.8 minutes for the IMAC purified as well as the affinity purified material. The retention time of hMN-14 Fab′, which has a molecular weight of approximately 50 kDa, was 9.75 minutes as indicated on the x-axis of FIG. 2B. The very similar retention time of hMN-14scFv indicates that it exists in solution as a dimer or diabody since the calculated molecular weight of the monomeric hMN-14scFv is 26 kDa. SDS-PAGE gel analysis (see FIG. 3A) shows a single band of the predicted size at 26 kDa, and the isoelectric focusing (IEF) gel analysis (see FIG. 3B) yields a band with pI of 8.2, close to the calculated pI of 7.9. A competitive ELISA showed that the hMN-14 diabody is functional and displays excellent binding properties.

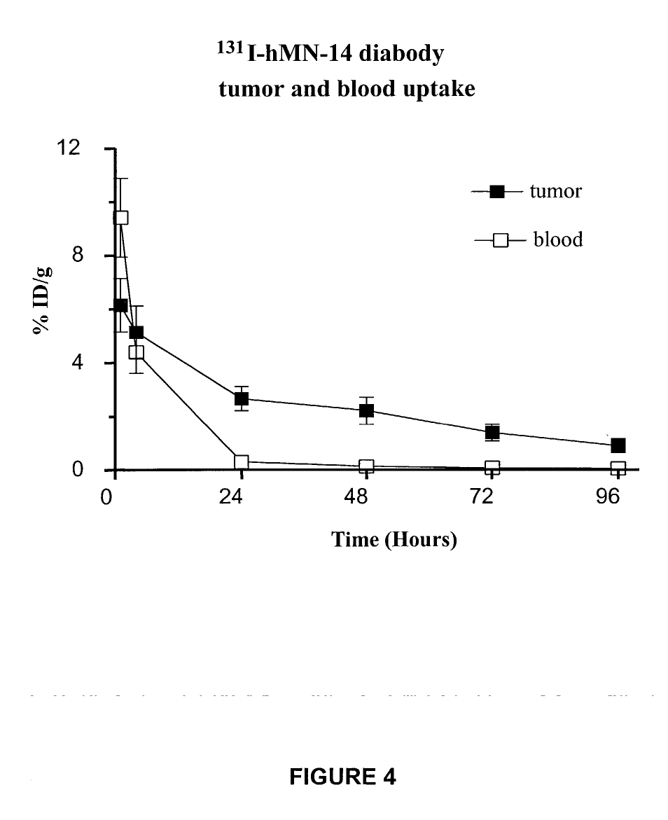

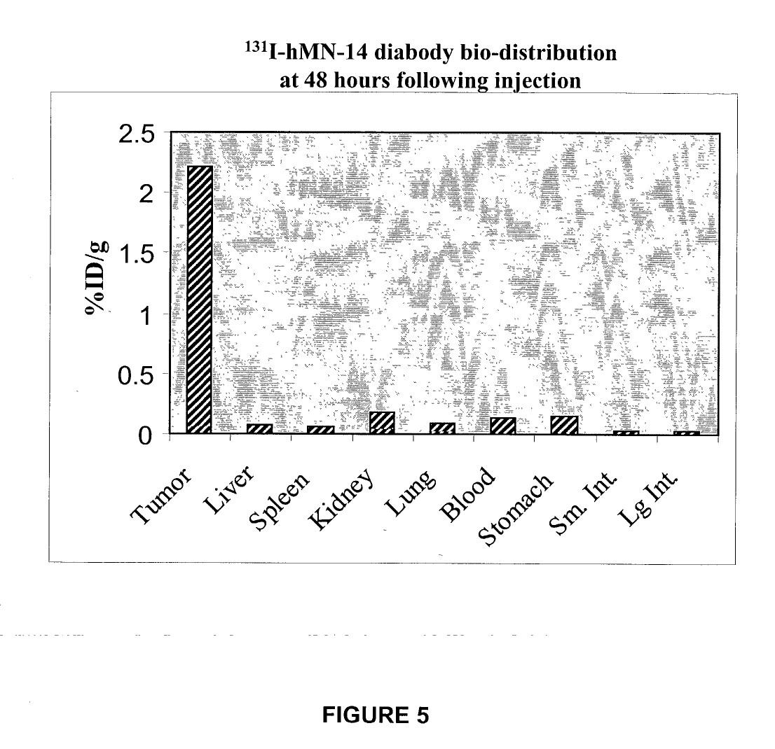

Nude mice bearing the CEA positive GW-39 tumor were injected with ¹³¹I-labeled hMN-14 diabody and the biodistribution was analyzed at various times following injection. While a significant amount of the diabody remained associated with the tumor for more than 96 hours, much of the free diabody cleared the blood rapidly as illustrated in FIG. 4. FIG. 5 shows the percentage of the injected dose that is associated with the tumor and with normal tissues, such as liver, spleen, kidney, lungs, blood, stomach, small intestine, and large intestine, at 48 hours after the injection. The amount of the injected dose in each normal tissue is very low when compared to the amount in the tumor. Table 1 summarizes the relative amounts of activity increased in the tumor over the listed normal tissues at 24, 48 and 72 hours (e.g., at 24 hours, the tumor has 22.47 times as much radioactivity as does the liver).

TABLE 1
Tumor to non-tumor ratios

| | 24 hours | 48 hours | 72 hours |
|---|---|---|---|
| Tumor | 1.00 | 1.00 | 1.00 |
| Liver | 22.47 | 31.85 | 28.32 |
| Spleen | 25.41 | 39.51 | 41.03 |
| Kidney | 9.12 | 12.12 | 10.54 |
| Lung | 15.49 | 25.70 | 31.75 |
| Blood | 9.84 | 17.32 | 21.80 |
| Stomach | 9.98 | 17.50 | 23.13 |
| Sm. Int. | 37.23 | 65.60 | 50.58 |
| Lg. Int. | 35.87 | 66.54 | 45.66 |

### Example 3

### Construction of a Plasmid for the Expression of hMN-14 Triabody

An hMN-14scFv plasmid construct, hMN-14-0, was designed, produced and tested. The E. coli expression plasmid directs the synthesis of a single polypeptide possessing the following features: (1) the carboxyl terminal end of hMN-14V_{H} is directly linked to the amino terminal end of hMN-14V_{K} without any additional amino acids (the use of the zero linker enables the secreted polypeptide to form a trimeric structure called a triabody, forming three binding sites for CEA); (2) a pelB signal peptide sequence precedes the V_{H} gene to facilitate the synthesis of the polypeptide in the periplasmic space of E. coli; and (3) six histidine (6His) residues are added to the carboxyl terminus to allow purification by IMAC. A schematic representation of the polypeptide and triabody are shown in FIG. 6.

Standard recombinant DNA methods were used to obtain the hMN-14-0 construct. The hMN-14 V_{H} and V_{K} sequences were amplified from the hMN-14scFv-L5 construct, using PCR with Pfu polymerase. The hMN-14V_{H} sequence was amplified using the oligonucleotide primers specified below:

| | |
|---|---|
| hMN-14V_{H}-Left | (SEQ ID NO:13) |

5′ - CGTACCATGGAGGTCCAACTGGTGGAGA - 3′

| | |
|---|---|
| hMN-14V_{H}-0 Right | (SEQ ID NO:14) |

5′ - GATATCGGAGACGGTGACCGGG - 3′

The left PCR primer, which was previously used for the construction of hMN-14scFv-L5, contains a 5′ NcoI restriction site. The right PCR primer contains EcoRV restriction site. The PCR product was cloned into PCR cloning vector pGemT (Promega).

The hMN-14V_{K} sequence was amplified using the oligonucleotide primers specified below:

| | |
|---|---|
| hMN-14V_{K}-0 Left | (SEQ ID NO:15) |

5′ - GATATCCAGCTGACCCAGAGCC - 3′

| | |
|---|---|
| hMN-14V_{K}-Right | (SEQ ID NO:16) |

5′ - GCTACTCGAGACGTTTGATTTCCACCTTGG - 3′

The left PCR primer contains an EcoRV restriction site. The right primer, which was previously used for the construction of hMN-14scFv-L5, contains an XhoI restriction site. The PCR product was cloned into pGemT vector. The V_{K}-0sequence was excised from the V_{K}-0-pGemT construct with EcoRV and SalI and ligated into the same sites of the V_{H}-0-pGemT construct to generate hMN-14-0 in pGemT. The V_{H}-V_{K} sequence was excised with NcoI and XhoI and transferred to pET26b to generate the hMN-14 triabody expression construct hMN-14-0. The DNA sequence of this construct was verified by automated DNA sequencing, and is listed in FIG. 13. The nucleic acid construct, hMN-14scFv-0, is illustrated in FIG. 6.

### Example 4

### Expression of hMN-14 Triabody in E. coli

The hMN-14-0 construct was used to transform BL21(P-LysS) E. coli. Culture conditions, induction, and purification were carried out similar to those described for the hMN-14 diabody in Example 2, except that the hMN-14-0 triabody was purified by Q-Sepharose anion exchange chromatography, instead of affinity chromatography. As expected, hMN-14-0 formed predominantly triabodies (˜80 kDa).

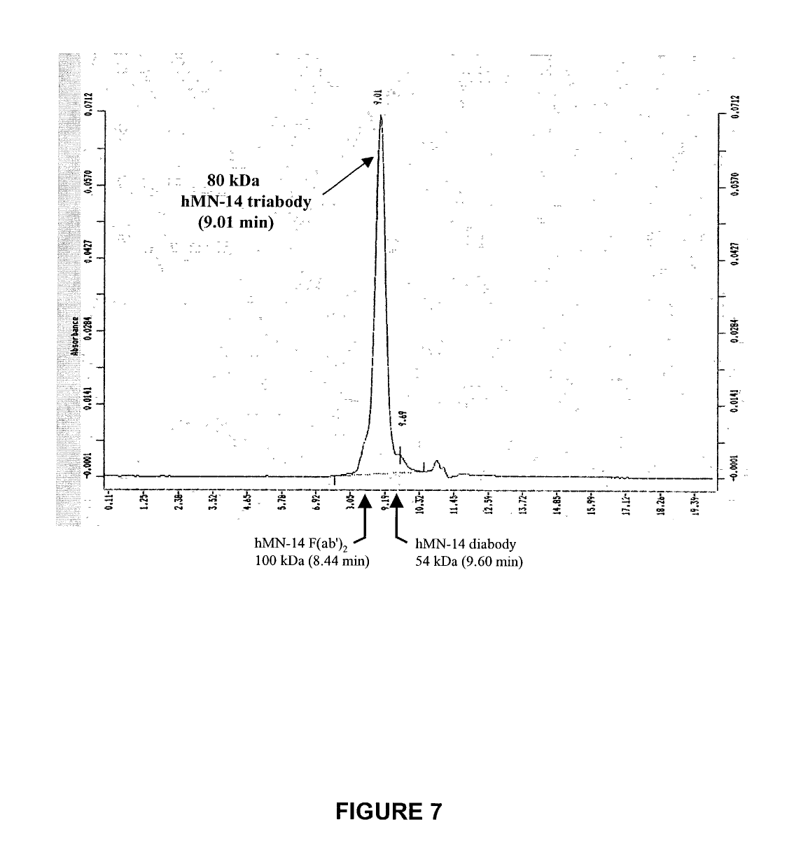

Approximately 2.4 mg/L culture of soluble hMN-14 triabody was purified from the soluble cell fraction of induced cultures. As shown by size-exclusion HPLC (see FIG. 7), a predominant peak was observed at 9.01 minutes for material purified by IMAC and mono-Q anion exchange chromatography. By comparison, the retention times of hMN-14 diabody (˜52 kDa) and hMN-14 F(ab′)2 (˜100 kDa) were 9.6 minutes and 8.44 minutes, respectively. The fact that the retention time of hMN-14-0 is exactly halfway between those of the 52 kDa and 100 kDa proteins indicates that it exists in solution as a trimer or triabody; since the calculated molecular weight of the monomeric hMN-14-0 polypeptide is ˜26 kDa. Indeed, SDS-PAGE analysis shows a single band of the predicted 26 kDa.

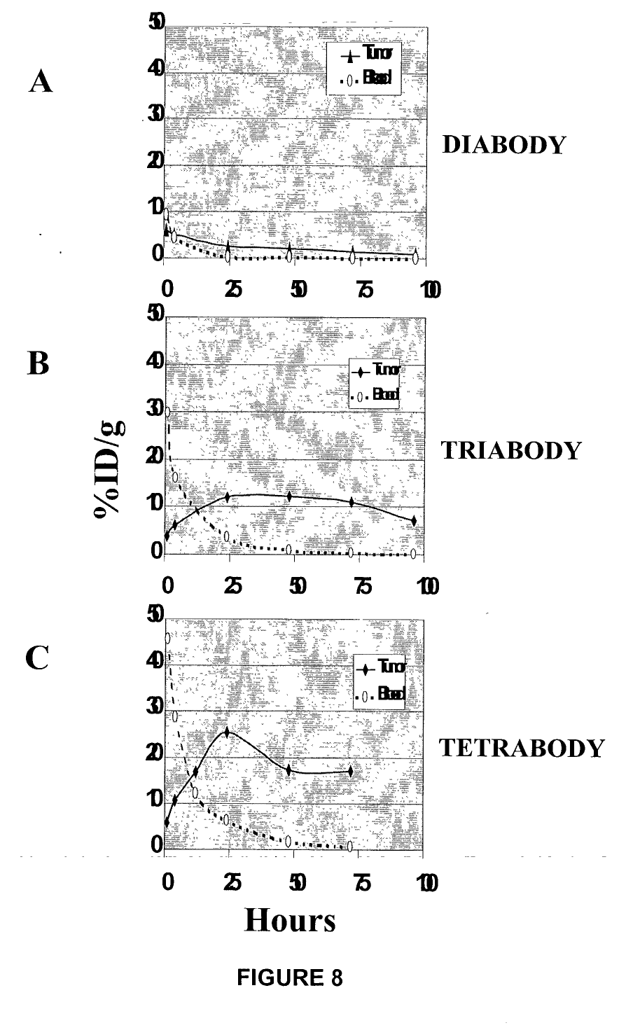

Nude mice bearing the CEA positive GW-39 tumor were injected with ¹³¹I-labeled hMN-14 triabody and the biodistribution was analyzed at various times following injection. FIG. 8 shows hMN-14 triabody tumor uptake and retention are remarkably higher than that of hMN-14 diabody. After one hour, triabody accumulates in the tumor at approximately 60% of the level of the diabody. However, while the diabody decreases steadily after one hour, triabody tumor uptake increases to a maximal level achieved between 24 and 48 hours. The maximal triabody tumor uptake (24-48 hours) is more than twice that of the diabody (1 hour). The tumor retention is also significantly longer for the triabody compared to diabody as the triabody may exhibit trivalent tumor binding by utilizing all three CEA binding sites. An additional factor that likely has a significant influence on tumor uptake is molecular size. As depicted in FIG. 8, blood clearance for the 80 kDa triabody is much slower than that of the 54 kDa diabody. This allows the triabody a much longier time to interact with the tumor. as compared to the diabody, and thus achieve higher levels of tumor uptake. The triabody's delayed blood clearance undoubtedly contributes to its superior tumor residence. However, other factors, including increased avidity due to multivalency or improved in vivo stability, may also contribute. Tumor to non-tumor ratios increased with time for all tissues (Table 2). The ratios were substantial at the later time points.

TABLE 2
Tumor to non tumor ratios for hMN-14 triabody.

| | 24 hours | 48 hours | 72 hours |
|---|---|---|---|
| Liver | 15.7 | 45.9 | 110.3 |
| Spleen | 13.7 | 39.9 | 96.9 |
| Kidney | 8.4 | 25.2 | 52.8 |
| Lung | 6.0 | 18.7 | 44.4 |
| Blood | 3.4 | 12.4 | 54.8 |
| Stomach | 11.3 | 15.0 | 62.4 |
| Sm. Int. | 28.3 | 78.5 | 204.7 |
| Lg Int. | 40.3 | 105.0 | 195.1 |

### Example 5

### Construction of Plasmids for the Expression of hMN-14 Tetrabodies

An hMN-14scFv plasmid construct, hMN-14-1G, was designed, produced and tested. The E. coli expression plasmid directs the synthesis of a single polypeptide possessing the following features: (1) the carboxyl terminal end of hMN-14V_{H} is linked to the amino terminal end of hMN-14V_{K} by a single glycine residue (the use of the 1G linker enables some of the secreted polypeptide to form a tetrameric structure called a tetrabody, forming four binding sites for CEA); (2) a pelB signal peptide sequence precedes the V_{H} gene to facilitate the synthesis of the polypeptide in the periplasmic space of E. coli; and (3) six histidine (6His) residues are added to the carboxyl terminus to allow purification by IMAC. A schematic representation of the polypeptide and tetrabody are shown in FIG. 9.

Standard recombinant DNA methods were used to obtain the hMN-14-1G construct. The hMN-14 V_{H} and V_{K} sequences were amplified from the hMN-14scFv-L5 construct, using PCR with Pfu polymerase. The hMN-14V_{H} sequence was amplified using the oligonucleotide primers specified below:

| | |
|---|---|
| hMN-14V_{H}-Left | (SEQ ID NO:17) |

5′ - CGTACCATGGAGGTCCAACTGGTGGAGA - 3′

| | |
|---|---|
| hMN-14V_{H}-1G Right | (SEQ ID NO:18) |

5′ - GCTGGATATCACCGGAGACGGTGACCGGGGTCC - 3′

The left PCR primer, which was previously used for the construction of hMN-14scFv-L5, contains a 5′ NcoI restriction site. The right PCR primer contains the coding sequence for a single glycine and an EcoRV restriction site. The PCR product was cloned into the PCR cloning vector pGemT (Promega). The hMN-14V_{K}-0 sequence (see Example 3) was excised from the hMN-14V_{K}-0-pGemT construct with EcoRV and SalI and ligated into the same sites of the hMN-14V_{H}-1G-pGemT construct to generate hMN-14-1G in pGemT. The V_{H}-LG-V_{K} sequence was excised with NcoI and XhoI and transferred to pET26b to generate the hMN-14 tetrabody expression construct hMN-14-1G. The DNA sequence of this construct was verified by automated DNA sequencing, and is listed in FIG. 14. The nucleic acid construct, hMN-14scFv-1G, is illustrated in FIG. 9.

### Example 6

### Expression of hMN-14 Tetrabodies in E. coli

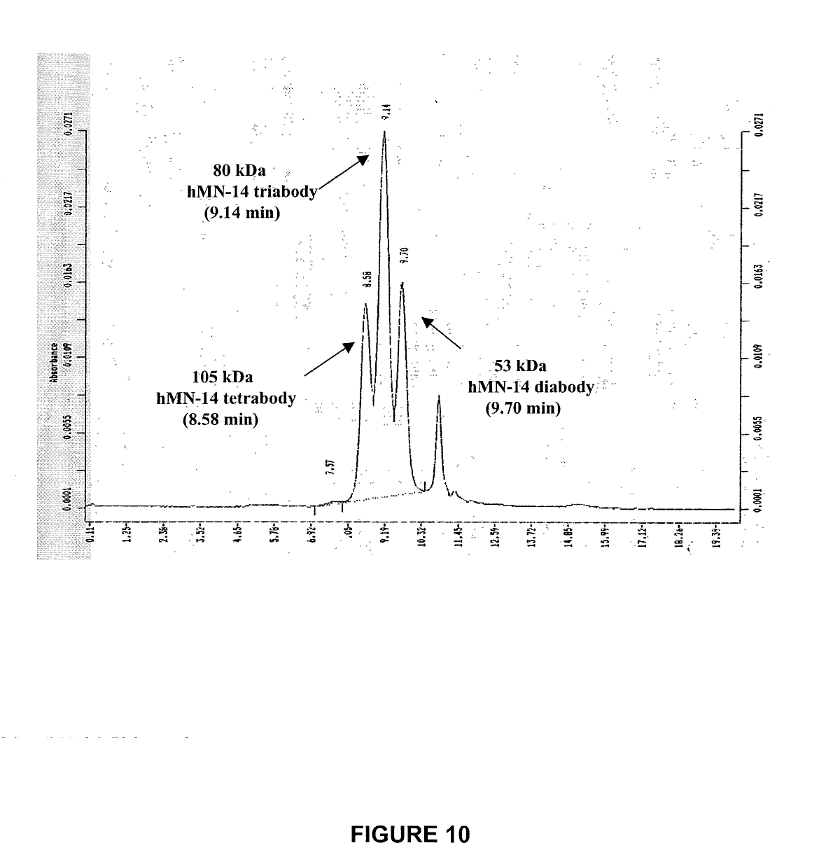

The hMN-14-1G construct was used to transform BL21(P-LysS) E. coli. Culture conditions, induction, and purification were carried out similar to those described for the hMN-14 diabody in Example 2, except that the hMN-14 tetrabody was purified by Q-Sepharose anion exchange chromatography, instead of affinity chromatography. Soluble expression levels were high, greater than 2 mg of soluble product was isolated per liter of culture. Size exclusion HPLC analysis (see FIG. 10) demonstrated that the hMN-14-1G product exists as a mixture of diabody (53 kDa), triabody (80 kDa) and tetrabody (105-120 kDa). The tetrabody could be isolated in relatively pure form by gel filtration chromatography. However, after several days at 2-8° C., it gradually reverted to a mixture of diabody, triabody and tetrabody similar to that shown in FIG. 10.

### Example 7

### Tumor Uptake of hMN-14 Diabody, Triabody, and Tetrabody

Tumor targeting was evaluated in mice bearing CEA-positive human colon tumor xenografts using radioiodinated samples. At 24 h, the diabody (obtained from hMN-14-L5) showed 2.7% injected dose per gram (ID/g) in the tumor, 0.3% in the blood, and 0.1 to 0.4% in all other organs. For the triabody (obtained from hMN-14-0), the tumor uptake was 12.0, 12.2, 11.1, and 7.1% ID/g at 24, 48, 72 and 96 h, respectively, with tumor to blood ratios increasing from 3.4 at 24 h to 12.4 at 48 h, and up to 55 at 96 h. The tetrabody (obtained from hMN-14-1G) displayed the highest tumor uptake among the three, reaching 25.4% ID/g at 24 h with a tumor to blood ratio of 3.9 and decreasing to 17.1% at 72 h, with a tumor to blood ratio of 29.3. These biodistribution results are in agreement with the respective molecular size and multivalency of the three novel scFv-based agents, all of which, and in particular the hMN-14 triabody, are especially useful for imaging and therapeutic applications.

While the invention has been described and exemplified in sufficient detail for those skilled in this art to make and use it, various alternatives, modifications, and improvements should be apparent without departing from the spirit and scope of the invention. The present invention is well adapted to carry out the objects and obtain the ends and advantages mentioned, as well as those inherent therein. The examples provided here are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

The disclosure of all publications cited above are expressly incorporated herein by reference in their entireties to the same extent as if each were incorporated by reference individually.

That which is claimed is:

1. A multivalent, monospecific binding protein comprising two or more binding sites having affinity for the same single target antigen, wherein said binding sites are formed by the association of two or more single chain Fv (scFv) fragments, and wherein each scFv fragment comprises at least 2 variable domains derived from a humanized or human monoclonal antibody.

2. The binding protein according to claim 1, wherein said monoclonal antibody is specific for a tumor-associated antigen.

3. The binding protein according to claim 2, wherein said tumor-associated antigen is associated with a disease state selected from the group consisting of a carcinoma, a melanoma, a sarcoma, a neuroblastoma, a leukemia, a glioma, a lymphoma and a myeloma.

4. The binding protein according to claim 2, wherein said tumor-associated antigen is associated with a type of cancer selected from the group consisting of acute lymphoblastic leukemia, acute myelogenous leukemia, biliary, breast, cervical, chronic lymphocytic leukemia, chronic myelogenous leukemia, colorectal, endometrial, esophageal, gastric, head and neck, Hodgkin's lymphoma, lung, medullary thyroid, non-Hodgkin's lymphoma, ovarian, pancreatic, prostrate, and urinary bladder.

5. The binding protein according to claim 2, wherein said tumor-associated antigen is selected from the group consisting of A3, A33, BrE3, CD1, CD1a, CD3, CD5, CD15, CD19, CD20D, CD21, CD22, CD23, CD30, CD45, CD74, CD79a, CEA, CSAp, EGFR, EGP-1, EGP-2, Ep-CAM, Ba 733, HER2/neu, KC4, KS-1, KS1-4, Le-Y, MAGE, MUC1, MUC2, MUC3, MUC4, PAM-4, PSA, PSMA, RS5, S100, T101, TAG-72, tenascin, Tn antigen, Thomson-Friedenreich antigens, tumor necrosis antigens, VEGF, 17-1A, an angiogenesis marker, a cytokine, an immunomodulator, an oncogene marker and an oncogene product.

6. The binding protein according to claim 2, wherein said tumor-associated antigen is carcinoembryonic antigen (CEA).

7. The binding protein according to claim 6, wherein the humanized monoclonal antibody is hMN-14.

8. The binding protein of claim 1, further comprising at least one agent selected from the group consisting of a diagnostic agent, a therapeutic agent, and combinations of two or more thereof.

9. The binding protein of claim 8, wherein said diagnostic agent is selected from the group consisting of a conjugate, a radionuclide, a metal, a contrast agent, a tracking agent, a detection agent, and combinations of two or more thereof.

10. The binding protein of claim 9, wherein said radionuclide is selected from the group consisting of ¹¹C, ¹³N, ¹⁵O, ¹⁸F, ³²P, ⁵¹Mn, ⁵²Fe, ^{52m}Mn, ⁵⁵Co, ⁶²Cu, ⁶⁴Cu, ₆₇Cu, ⁶⁷Ga, ⁶⁸Ga, ⁷²As, ⁷⁵Br, ⁷⁶Br, ^{82m}Rb, ⁸³Sr, ⁸⁶Y, ⁸⁹Zr, ⁹⁰Y, ^{94m}Tc, ⁹⁴Tc, ^{99m}Tc, ¹¹⁰In, ¹¹¹In, ¹²⁰I, ¹²³I, ¹²⁴I, ¹²⁵I, ¹³¹I, ¹⁵⁴⁻¹⁵⁸Gd, ¹⁷⁷Lu, ¹⁸⁶Re, ¹⁸⁸Re, a gamma-emitter, a beta-emitter, a positron-emitter, and combinations of two or more thereof.

11. The binding protein of claim 9, wherein said radionuclide is selected from the group consisting of ⁵¹Cr, ⁵⁷Co, ⁵⁸Co, ⁵⁹Fe, ⁶⁷Cu, ⁶⁷Ga, ⁷⁵Se, ⁹⁷Ru, ^{99m}Tc, ¹¹¹In, ^{114m}In, ¹²³I, ¹²⁵I, ¹³¹I, ¹⁶⁹Yb, ¹⁹⁷Hg, ²⁰¹Tl, and combinations of two or more thereof.

12. The binding protein of claim 9, wherein said metal is selected from the group consisting of gadolinium, iron, chromium, copper, cobalt, nickel, dysprosium, rhenium, europium, terbium, holmium, neodymium, and combinations of two or more thereof.

13. The binding protein of claim 9, wherein said contrast agent is a MRI contrast agent.

14. The binding protein of claim 9, wherein said contrast agent is a CT contrast agent.

15. The binding protein of claim 9, wherein said contrast agent is an ultrasound contrast agent.

16. The binding protein of claim 9, wherein said contrast agent is selected from the group consisting of agadolinium ions, lanthanum ions, manganese ions, iron, chromium, copper, cobalt, nickel, dysporsium, rhenium, europium, terbium, holmium, neodymium, another comparable contrast agent, and combinations of two or more thereof.

17. The binding protein of claim 9, wherein said tracking agent is selected from the group consisting of iodine compounds, barium compounds, gallium compounds, thallium compounds, barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, and combinations of two more thereof.

18. The binding protein of claim 9, wherein said detection agent is selected from the group consisting of an enzyme, a fluorescent compound, a chemiluminescent compound, a bioluminescent compound, a radioisotope, and combinations of two or more thereof.

19. The binding protein of claim 8, wherein said therapeutic agent is selected from the group consisting of a radionuclide, a chemotherapeutic drug, a cytokine, a hormone, a growth factor, a toxin, an immunomodulator, and combinations of two or more thereof.

20. The binding protein of claim 19, wherein said radionuclide is selected from the group consisting of ³²P, ³³P, ⁴⁷Sc, ⁵⁹Fe, ⁶²Cu, ⁶⁴Cu, ⁶⁷Cu, ⁶⁷Ga, ⁷⁵Se, ⁷⁷As, ⁸⁹Sr, ⁹⁰Y, ⁹⁹Mo, ¹⁰⁵Rh, ¹⁰⁹Pd, ¹¹¹Ag, ¹¹¹In, ¹²⁵I, ¹³¹I, ¹⁴²Pr, ¹⁴³Pr, ¹⁴⁹Pm, ¹⁵³Sm, ¹⁶¹Tb, ¹⁶⁶Dy, ¹⁶⁶Ho, ¹⁶⁹Er, ¹⁷⁷Lu, ¹⁸⁶Re, ¹⁸⁸Re, ¹⁸⁹Re, 194Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, and combinations of two or more thereof.

21. The binding protein of claim 19, wherein said radionuclide is selected from the group consisting of ⁵⁸Co, ⁶⁷Ga, ^{80m}Br, ^{99m}Tc, ^{103m}Rh, ¹⁰⁹Pt, ¹¹¹In, ¹¹⁹Sb, ¹²⁵I, ¹⁶¹Ho, ^{189m}Os and ¹⁹²Ir, ¹⁵²Dy, ²¹¹At, ²¹¹Bi, ²¹²Bi, ²¹³Bi, ²¹⁵Po, ²¹⁷At, ²¹⁹Rn, ²²¹Fr, ²²³Ra, ²²⁵Ac, ²⁵⁵Fm, and combinations of two or more thereof.

22. The binding protein of claim 19, wherein said chemotherapeutic drug is selected from the group consisting of vinca alkaloids, anthracyclines, epidophyllotoxins, taxanes, antimetabolites, alkylating agents, antibiotics, Cox-2 inhibitors, antimitotics, antiangiogenic agents, apoptotoic agents, doxorubicin, methotrexate, taxol, CPT-11, camptothecans, nitrogen mustards, alkyl sulfonates, nitrosoureas, triazenes, folic acid analogs, pyrimidine analogs, purine analogs, platinum coordination complexes, hormones, and combinations of two or more thereof.

23. The binding protein of claim 19, wherein said toxin is selected from the group consisting of ricin, abrin, ribonuclease, DNase I, Staphylococcal enterotoxin A, pokeweed antiviral protein, gelonin, diphtherin toxin, Pseudomonas exotoxin, Pseudomonas endotoxin, and combinations of two or more thereof.

24. The binding protein of claim 19, wherein said immunomodulator is selected from the group consisting of cytokines, stem cell growth factors, lymphotoxins, hematopoietic factors, colony stimulating factors, interferons, stem cell growth factors, erythropoietin, thrombopoietin, and combinations of two or more thereof.

25. A multivalent, monospecific binding protein comprising two binding sites having affinity for the same single target antigen (termed a monospecific diabody), wherein said binding sites are formed by the association of two single chain Fv (scFv) fragments, and wherein each scFv fragment comprises at least 2 variable domains derived from a humanized or human monoclonal antibody.

26. The monospecific diabody according to claim 25, wherein said monoclonal antibody is specific for a tumor-associated antigen.

27. The monospecific diabody according to claim 26, wherein said tumor-associated antigen is carcinoembryonic antigen (CEA).

28. The monospecific diabody according to claim 25, wherein the humanized monoclonal antibody is hMN-14.

29. The monospecific diabody according to claim 28, wherein each scFv comprises the V_{H} and the V_{K} regions of hMN-14.

30. The monospecific diabody according to claim 29, wherein each scFv further comprises an amino acid linker connecting the V_{H} and the V_{K} regions of hMN-14.

31. The monospecific diabody according to claim 30, wherein each scFv comprises the amino acid sequence of SEQ ID NO: 2.

32. An expression vector comprising a nucleotide sequence encoding the monospecific diabody of claim 25.

33. A host cell comprising the expression vector of claim 32.

34. A multivalent, monospecific binding protein comprising three binding sites having affinity for the same single target antigen (termed a monospecific triabody), wherein said binding sites are formed by the association of three single chain Fv (scFv) fragments, and wherein each scFv fragment comprises at least 2 variable domains derived from a humanized or human monoclonal antibody.

35. The monospecific triabody according to claim 34, wherein said monoclonal antibody is specific for a tumor-associated antigen.

36. The monospecific triabody according to claim 35, wherein said tumor-associated antigen is carcinoembryonic antigen (CEA).

37. The monospecific triabody according to claim 34, wherein the humanized monoclonal antibody is hMN-14.

38. The monospecific triabody according to claim 37, wherein each scFv comprises the V_{H} and the V_{K} regions of hMN-14.

39. The monospecific triabody according to claim 38, wherein each scFv comprises the amino acid sequence of SEQ ID NO: 6.

40. An expression vector comprising a nucleotide sequence encoding the monospecific triabody of claim 34.

41. A host cell comprising the expression vector of claim 40.

42. A multivalent, monospecific binding protein comprising four binding sites having affinity for the same single target antigen (termed a monospecific tetrabody), wherein said binding sites are formed by the association of four single chain Fv (scFv) fragments, and wherein each scFv fragment comprises at least 2 variable domains derived from a humanized or human monoclonal antibody.

43. The monospecific tetrabody according to claim 42, wherein said monoclonal antibody is specific for a tumor-associated antigen.

44. The monospecific tetrabody according to claim 43, wherein said tumor-associated antigen is carcinoembryonic antigen (CEA).

45. The monospecific tetrabody according to claim 42, wherein the humanized monoclonal antibody is hMN-14.

46. The monospecific tetrabody according to claim 45, wherein each scFv comprises the V_{H} and the V_{K} regions of hMN-14.

47. The monospecific tetrabody according to claim 46, wherein each scFv further comprises an amino acid linker connecting the V_{H} and the V_{K} regions of hMN-14.

48. The monospecific tetrabody according to claim 47, wherein each scFv comprises the amino acid sequence of SEQ ID NO: 8.

49. An expression vector comprising a nucleotide sequence encoding the monospecific tetrabody of claim 42.

50. A host cell comprising the expression vector of claim 49.

51. A method of diagnosing the presence of a tumor, said method comprising administering to a subject suspected of having a tumor a detectable amount of the binding protein of claim 9, and monitoring the subject to detect any binding of the binding protein to a tumor.

52. A method of treating a tumor, said method comprising administering to a subject in need thereof an effective amount of the binding protein of claim 19.

53. A method of diagnosing the presence of a tumor, said method comprising administering to a subject suspected of having a tumor a detectable amount of the binding protein of claim 1 in combination with a detectable moiety that is capable of binding to said binding protein, and monitoring the subject to detect any binding of the binding protein to a tumor.

54. A method of treating a tumor, said method comprising administering to a subject in need thereof an effective amount of the binding protein of claim 1 in combination with a therapeutic agent.

55. A method according to claim 54, wherein said therapeutic agent is selected from the group consisting of a chemotherapeutic drug, a toxin, external radiation, a brachytherapy radiation agent, a radiolabeled protein, an anticancer drug and an anticancer antibody.

56. A method of delivering one or more diagnostic agent, one or more therapeutic agent, or a combination of two or more thereof to a tumor, said method comprising administering to a subject in need thereof the binding protein of claim 8.

57. A kit for therapeutic and/or diagnostic use, said kit comprising at least one binding protein according to claim 8, and additional reagents, equipment, and instructions for use.
